(19) United States
(12) Patent Application Publication
(10) Pub. No.: US 2026/0259346 A1
(43) Pub. Date: Sep. 3, 2026

(54) METHOD AND DEVICE FOR EVALUATING PRODUCTION PROPORTIONS OF DIFFERENT GASES IN DEEP COALBED METHANE RESERVOIR

(71) Applicants: Xi’an Shiyou University, Xi'an City (CN); China University of Petroleum, Beijing, Beijing (CN)

(72) Inventors: Juntai SHI, Beijing (CN); Hai HUANG, Xi'an City (CN); Xianyue XIONG, Beijing (CN); Ming CHEN, Wuhan City (CN); Hongxing HUANG, Beijing (CN); Wentong ZHANG, Xianyang City (CN); Kai WANG, Xi'an City (CN); Shuo ZHANG, Nenjiang City (CN); Yitianyu JI, Neijiang City (CN)

(21) Appl. No.: 19/444,712

(22) Filed: Jan. 9, 2026

(30) Foreign Application Priority Data
Feb. 28, 2025 (CN) 202510233898.5

(51) Int. Cl. G01V 99/00

(52) U.S. Cl. CPC G01V 99/00

(57) ABSTRACT

[symbol]

vided are a method and device for evaluating production proportions of different gases in [symbol] coalbed methane reservoir. It includes: calculating a coalbed porosity and a coalbed water [symbol] on at the beginning of production following fracturing flowback based on the physical [symbol] y parameters of the target coalbed methane reservoir; calculating original reserves of [symbol] ed gas, free gas, and dissolved gas based on the physical property parameters of the target coalbed methane reservoir; calculating an average coalbed pressure and cumulative productions of the three gases each day based on cumulative quantities of gas production, water production, and water influx each day, the coalbed porosity and the coalbed water saturation at the beginning of production following fracturing flowback, and the physical property parameters; and calculating daily production rates of the three gases based on the cumulative production of each gas, obtaining daily production proportions of the three gases.

### CROSS REFERENCE TO RELATED APPLICATION

This patent application claims the benefit and priority of Chinese Patent Application No. 2025102338985, filed with the China National Intellectual Property Administration on Feb. 28, 2025, the disclosure of which is incorporated by reference herein in its entirety as part of the present application.

### TECHNICAL FIELD

The present application relates to the technical field of coalbed methane reservoir development, and in particular, to a method and device for evaluating production proportions of different gases in a deep coalbed methane reservoir.

### BACKGROUND

As one of important unconventional resources, target coalbed methane reservoirs have attracted widespread global attention due to their potential to alleviate energy shortages, reduce coal mine safety accidents, and mitigate environmental stress. In recent years, the development of target coalbed methane reservoirs has progressed rapidly, with a plurality of target coalbed methane reservoir blocks achieving breakthroughs in high production. This is attributed to the fact that the gas types in target coalbed methane reservoirs not only include adsorbed gas but are also rich in free gas, along with a certain amount of dissolved gas. Studying content proportions of gases of different types in a target coalbed methane reservoir is of significant theoretical and practical importance for evaluating coalbed methane reserves, predicting geological sweet spots, and the like.

Currently, there are various methods for determining content proportions (i.e., reserve proportions) of gases of different types in a target coalbed methane reservoir, but fewer methods for evaluating production proportions of gases of different types in a target coalbed methane reservoir. Only a small number of studies have established evaluation methods for the adsorbed gas/free gas ratios in coalbed methane wells based on carbon isotope fractionation models. These methods require the installation of carbon isotope monitoring devices at wellheads, which limits their applicability, and fail to account for the production proportion of the dissolved gas. Thus, methods that rely solely on coalbed methane well production data to evaluate production proportions of gases of different types in a target coalbed methane reservoir are rarely reported.

### SUMMARY

An objective of embodiments of the present application is to provide a method and device for evaluating production proportions of different gases in a deep coalbed methane reservoir to address the technical problems of significant errors and high prediction costs in evaluating the production proportions of different gases in the deep coalbed methane reservoir.

To achieve the above objective, a first aspect of the present application provides a method for evaluating production proportions of different gases in a deep coalbed methane reservoir, including following steps:
obtaining physical property parameters of a target coalbed methane reservoir;
calculating, after fracturing flowback and before production, a coalbed porosity and a coalbed water saturation at the beginning of production of the target coalbed methane reservoir after the fracturing flowback based on the physical property parameters of the target coalbed methane reservoir;
calculating an original adsorbed gas reserve, an original free gas reserve, and an original dissolved gas reserve of the target coalbed methane reservoir based on the physical property parameters of the target coalbed methane reservoir, the coalbed porosity, and the coalbed water saturation;
calculating an average coalbed pressure value of the target coalbed methane reservoir each day based on the original adsorbed gas reserve, the original free gas reserve, and the original dissolved gas reserve of the target coalbed methane reservoir, a cumulative gas production, a cumulative water production, and a cumulative water influx each day, the coalbed porosity and the coalbed water saturation at the beginning of production of the target coalbed methane reservoir after the fracturing flowback, and the physical property parameters;
calculating a cumulative free gas production, a cumulative adsorbed gas production, and a cumulative dissolved gas production of the target coalbed methane reservoir based on the average coalbed pressure value of the target coalbed methane reservoir each day, the original adsorbed gas reserve, the original free gas reserve, the original dissolved gas reserve, the cumulative gas production, the cumulative water production, and the cumulative water influx each day, the coalbed porosity and the coalbed water saturation at the beginning of production of the target coalbed methane reservoir after the fracturing flowback, and the physical property parameters;
calculating a daily free gas production, a daily adsorbed gas production, and a daily dissolved gas production of the target coalbed methane reservoir based on the cumulative free gas production, the cumulative adsorbed gas production, and the cumulative dissolved gas production of the target coalbed methane reservoir each day; and
calculating daily production proportions of three gases in the target coalbed methane reservoir based on the daily free gas production, the daily adsorbed gas production, and the daily dissolved gas production.

In an embodiment of the present disclosure, the step of calculating, after fracturing flowback and before production, a coalbed porosity and a coalbed water saturation at the beginning of production of the target coalbed methane reservoir after the fracturing flowback based on the physical property parameters of the target coalbed methane reservoir includes:
obtaining a total fracturing fluid quantity during a fracturing process of the target coalbed methane reservoir and a cumulative flowback quantity during a flowback process;
calculating a net fracturing injection quantity based on the total fracturing fluid quantity and the cumulative flowback quantity;
calculating the coalbed porosity at the beginning of production of the target coalbed methane reservoir after the fracturing flowback based on a coalbed controlled volume, the net fracturing injection quantity, an initial porosity of the target coalbed methane reservoir, and a volume factor of water; and
calculating the coalbed water saturation based on the coalbed controlled volume, the initial porosity of the target coalbed methane reservoir, an initial water saturation, the net fracturing injection quantity, the volume factor of water, and the coalbed porosity at the beginning of production of the target coalbed methane reservoir after the fracturing flowback.

In an embodiment of the present application, the step of calculating an average coalbed pressure value of the target coalbed methane reservoir each day based on the original adsorbed gas reserve, the original free gas reserve, and the original dissolved gas reserve of the target coalbed methane reservoir, a cumulative gas production, a cumulative water production, and a cumulative water influx each day, the coalbed porosity and the coalbed water saturation at the beginning of production of the target coalbed methane reservoir after the fracturing flowback, and the physical property parameters includes:
establishing a material balance equation for adsorbed gas in adsorption pores according to the material balance principle for adsorbed gas desorption;
establishing a material balance equation for free gas and dissolved gas in free pores according to the material balance principle for the free gas and the dissolved gas;
establishing a material balance equation for the target coalbed methane reservoir according to the material balance equation for the adsorbed gas and the material balance equation for the free gas and the dissolved gas; and
substituting the original adsorbed gas reserve, the original free gas reserve, and the original dissolved gas reserve of the target coalbed methane reservoir, the cumulative gas production, the cumulative water production, the cumulative water influx, the coalbed porosity and the coalbed water saturation at the beginning of production of the target coalbed methane reservoir after the fracturing flowback, and the physical property parameters into the material balance equation for the target coalbed methane reservoir, and calculating the average coalbed pressure value by one of a Newton iteration method, a secant method, and a univariate cubic function calculation method.

In an embodiment of the present application, the calculating the average coalbed pressure value by a univariate cubic function calculation method includes:
establishing a univariate cubic function model according to the material balance equation for the target coalbed methane reservoir;
calculating a quadratic factor, a linear factor, and a constant term of the univariate cubic function model based on the original adsorbed gas reserve, the original free gas reserve, and the original dissolved gas reserve of the target coalbed methane reservoir, the cumulative gas production, the cumulative water production, and the cumulative water influx each day, the coalbed porosity and the coalbed water saturation at the beginning of production of the target coalbed methane reservoir after the fracturing flowback, and the physical property parameters; and
calculating the average coalbed pressure value according to the quadratic factor, the linear factor, and the constant term of the univariate cubic function model.

In an embodiment of the present application, the cumulative free gas production of the target coalbed methane reservoir is calculated by a formula below:

$${G}_{p f} = {G}_{fi} - \frac{V {ϕ}_{i p} {Z}_{s c} {T}_{s c} p}{{p}_{s c} T Z} [ 1 - {S}_{w i p} - ( {C}_{p} + {S}_{w i p} {C}_{w} ) ( {p}_{i} - p ) - \frac{{W}_{e} - {W}_{p} {B}_{w}}{V {ϕ}_{i p}} + {c}_{a} ( \frac{{p}_{i} + {p}_{c}}{{p}_{L} + {p}_{i} + {p}_{c}} - \frac{p + {p}_{c}}{{p}_{L} + p + {p}_{c}} ) ]$$

where G_{pf} represents the cumulative free gas production of the target coalbed methane reservoir each day, 10⁶ m³; G_{fi} represents an original free gas reserve controlled by the target coalbed methane reservoir; Wₚ represents a cumulative water production of the target coalbed methane reservoir, 10⁶ m³; Wₑ represents a cumulative water influx during a production process, 10⁶ m³; φᵢₚ represents the coalbed porosity at the beginning of production of the target coalbed methane reservoir after the fracturing flowback; V represents a coalbed controlled volume, 10⁶ m³; S_{wip} represents the coalbed water saturation at the beginning of production of the target coalbed methane reservoir after the fracturing flowback; p_{sc} represents a pressure under standard conditions, MPa, with a value of 0.101325; T_{sc} represents a temperature under standard conditions, K, with a value of 293.15; Z_{sc} represents a natural gas deviation factor under standard conditions, dimensionless, with a value of 1; T represents a target coalbed temperature, K; Zᵢ represents a gas deviation factor under an initial coalbed pressure, dimensionless; p_{L} represents Langmuir pressure, MPa; p_{c} represents a capillary pressure between coalbed micropores and mesopores, MPa; pᵢ represents the initial coalbed pressure, MPa; p represents an average coal reservoir pressure, MPa; Z represents a gas deviation factor under an average coalbed pressure, dimensionless; Cₚ represents a pore volume compressibility, MPa⁻¹; C_{w} represents an isothermal compressibility of water, MPa⁻¹; Cₛ represents a dissolution factor of coalbed methane in water, MPa⁻¹; B_{w} represents a volume factor of water, m³/m³; and Cₐ represents a coal matrix shrinkage factor, dimensionless.

In an embodiment of the present application, the cumulative dissolved gas production of the target coalbed methane reservoir is calculated by a formula below:

$${G}_{p s} = {G}_{s i} - V {ϕ}_{i p} {C}_{S} p [ {S}_{w i p} + {S}_{w i p} {C}_{w} ( {p}_{i} - p ) + \frac{{W}_{e} - {W}_{p} {B}_{w}}{V {ϕ}_{i p}} ]$$

where Gₚₛ represents the cumulative dissolved gas production of the target coalbed methane reservoir each day, 10⁶ m³; Gₛᵢ represents an original dissolved gas reserve controlled by the target coalbed methane reservoir; Wₚ represents a cumulative water production of the target coalbed methane reservoir, 10⁶ m³; Wₑ represents a cumulative water influx during a production process, 10⁶ m³; φᵢₚ represents the coalbed porosity at the beginning of production of the target coalbed methane reservoir after the fracturing flowback; V represents a coalbed controlled volume, 10⁶ m³; S_{wip} represents the coalbed water saturation at the beginning of production of the target coalbed methane reservoir after the fracturing flowback; pᵢ represents an initial coalbed pressure, MPa; p represents an average coal reservoir pressure, MPa; Cₛ represents a dissolution factor of coalbed methane in water, MPa⁻¹; B_{w} represents a volume factor of water, m³/m³; and C_{w} represents an isothermal compressibility of water, MPa⁻¹.

In an embodiment of the present application, the cumulative adsorbed gas production of the target coalbed methane reservoir is calculated by a formula below:

$${G}_{p a} = V {ρ}_{c} \frac{{V}_{L} ( {p}_{i} + {p}_{c} )}{{p}_{L} + {p}_{i} + {p}_{c}} - V {ρ}_{c} \frac{{V}_{L} ( p + {p}_{c} )}{{p}_{L} + p + {p}_{c}}$$

where Gₚₐ represents a cumulative desorption quantity of adsorbed gas in the target coalbed methane reservoir into free pores, namely the cumulative adsorbed gas production, 10⁶ m³; ρ_{c} represents a coal rock density, t/m³; V_{L} represents Langmuir volume, m³/t; p_{L} represents Langmuir pressure, MPa; p_{c} represents a capillary pressure between target coalbed micropores and mesopores, MPa; pᵢ represents an initial coalbed pressure, MPa; and p represents an average coal reservoir pressure, MPa.

In an embodiment of the present application, the daily free gas production of the target coalbed methane reservoir is calculated by a formula below:

$${q}_{pf , j} = \{ \begin{matrix}{10}^{6} \frac{{G}_{pf , j} - {G}_{pf , j - 1}}{1} , & {G}_{pf , j} > {G}_{pf , j - 1} \\ 0 , & {G}_{pf , j} \leq {G}_{pf , j - 1}\end{matrix}$$

where q_{pf,j} represents the daily free gas production of the target coalbed methane reservoir on a current day (day j), m³/d; G_{pf,j} represents the cumulative free gas production of the target coalbed methane reservoir on the current day (day j), 10⁶ m³; and G_{pf,j-1} represents the cumulative free gas production of the target coalbed methane reservoir on a previous day (day j-1), 10⁶ m³.

In an embodiment of the present application, the daily adsorbed gas production is calculated by a formula below:

$${q}_{pa , j} = \{ \begin{matrix}{10}^{6} \frac{{G}_{p a , j} - {G}_{p a , j - 1}}{1} , & {G}_{p f , j} > {G}_{p f , j - 1} \\ {10}^{6} \frac{{G}_{p a , j} - {G}_{p a , j - 1} + {G}_{p f , j} - {G}_{p f , j - 1}}{1} , & {G}_{p f , j} \leq {G}_{p f , j - 1}\end{matrix}$$

where q_{pa,j} represents the daily adsorbed gas production of the target coalbed methane reservoir on a current day (day j), m³/d; G_{pa,j} represents the cumulative adsorbed gas production of the target coalbed methane reservoir on the current day (day j), 10⁶ m³; and Gₚₐⱼ₋₁ represents the cumulative adsorbed gas production of the target coalbed methane reservoir on a previous day (day j-1), 10⁶ m³; and
the daily dissolved gas production is calculated by a formula below:

$${q}_{p s , j} = 1 {0}^{6} \frac{{G}_{p s , j} - {G}_{p s , j - 1}}{1}$$

where q_{ps,j} represents the daily dissolved gas production of the target coalbed methane reservoir on the current day (day j), m³/d; G_{ps,j} represents the cumulative dissolved gas production of the target coalbed methane reservoir on the current day (day j), 10⁶ m³; and G_{ps,j-1} represents the cumulative dissolved gas production of the target coalbed methane reservoir on the previous day (day j-1), 10⁶ m³.

A second aspect of the present application provides a device for evaluating production proportions of different gases in a deep coalbed methane reservoir, including:
a memory configured to store instructions; and
a processor configured to invoke the instructions from the memory and implement the method for evaluating production proportions of different gases in a deep coalbed methane reservoir described above when executing the instructions.

With the above technical solutions, when the production proportions of gases are calculated, the physical property parameters of the target coalbed methane reservoir are obtained first. After fracturing flowback and before production, the coalbed porosity and the coalbed water saturation at the beginning of production of the target coalbed methane reservoir after the fracturing flowback are calculated based on the physical property parameters of the target coalbed methane reservoir. The original adsorbed gas reserve, the original free gas reserve, and the original dissolved gas reserve of the target coalbed methane reservoir are calculated based on the physical property parameters of the target coalbed methane reservoir, the coalbed porosity, and the coalbed water saturation. The average coalbed pressure value of the target coalbed methane reservoir each day is calculated based on the original adsorbed gas reserve, the original free gas reserve, and the original dissolved gas reserve of the target coalbed methane reservoir, the cumulative gas production, the cumulative water production, and the cumulative water influx each day, the coalbed porosity and the coalbed water saturation at the beginning of production of the target coalbed methane reservoir after the fracturing flowback, and the physical property parameters. The cumulative free gas production, the cumulative adsorbed gas production, and the cumulative dissolved gas production of the target coalbed methane reservoir are calculated based on the average coalbed pressure value of the target coalbed methane reservoir each day, the original adsorbed gas reserve, the original free gas reserve, and the original dissolved gas reserve of the target coalbed methane reservoir, the cumulative gas production, the cumulative water production, and the cumulative water influx each day, the coalbed porosity and the coalbed water saturation at the beginning of production of the target coalbed methane reservoir after the fracturing flowback, and the physical property parameters. The daily free gas production, the daily adsorbed gas production, and the daily dissolved gas production of the target coalbed methane reservoir are calculated based on the cumulative free gas production, the cumulative adsorbed gas production, and the cumulative dissolved gas production of the target coalbed methane reservoir each day. The daily production proportions of the three gases in the target coalbed methane reservoir are calculated based on the daily free gas production, the daily adsorbed gas production, and the daily dissolved gas production. The method for evaluating production proportions of adsorbed gas, free gas, and dissolved gas in a deep coalbed methane reservoir provided herein requires fewer parameters, eliminates the need for installing a carbon isotope monitoring device at a wellhead, and is applicable to all deep coalbed methane reservoirs, allowing for large-scale promotion and use.

Other features and advantages of the embodiments of the present application are described in detail in the following DETAILED DESCRIPTION OF THE EMBODIMENTS part.

### BRIEF DESCRIPTION OF THE DRAWINGS

The accompanying drawings are provided for further understanding of the embodiments of the present application, and constitute a part of the specification. The accompanying drawings and the following specific implementations are intended to explain the embodiments of the present application, rather than to limit the embodiments of the present application. In the drawings:

FIG. 1 diagrammatically illustrates a flowchart of a method for evaluating production proportions of different gases in a deep coalbed methane reservoir according to an embodiment of the present application;

FIG. 2 diagrammatically illustrates a curve graph of a cumulative gas production Gₚ and a cumulative water production Wₚ of a deep coalbed methane reservoir each day;

FIG. 3 diagrammatically illustrates a curve graph of an average coal reservoir pressure p of a deep coalbed methane reservoir each day;

FIG. 4 diagrammatically illustrates a curve graph of a cumulative gas production, a cumulative adsorbed gas production, a cumulative free gas production, and a cumulative dissolved gas production of a deep coalbed methane reservoir each day;

FIG. 5 diagrammatically illustrates a daily gas production, a daily adsorbed gas production, a daily free gas production, and a daily dissolved gas production of a deep coalbed methane reservoir each day; and

FIG. 6 diagrammatically illustrates a curve graph of a daily adsorbed gas production proportion, a daily free gas production proportion, and daily dissolved gas production proportion in a deep coalbed methane reservoir each day.

### DETAILED DESCRIPTION OF THE EMBODIMENTS

In order to make the objectives, technical solutions, and advantages of the embodiments of the present application clearer, the technical solutions in the embodiments of the present application are clearly and completely described below with reference to the accompanying drawings in the embodiments of the present application. Apparently, the described specific implementations are intended to describe and explain the embodiments of the present application, rather than to limit the embodiments of the present application. All other embodiments obtained by those of ordinary skill in the art based on the embodiments of the present application without creative efforts should fall within the protection scope of the present application.

It should be noted that, in the technical solutions of the present application, the acquisition, transmission, storage, use, processing, etc., of data all comply with the relevant provisions of national laws and regulations of China. In the embodiments of the present application, existing industry solutions such as certain software, components, and models may be mentioned. These should be considered exemplary, and their purpose is solely to illustrate the feasibility of implementing the technical solutions of the present application. However, this does not imply that the applicant has already used or necessarily intends to use such solutions.

Moreover, the terms such as “first” and “second” described in the embodiments of the present application are used herein only for the purpose of description and are not intended to indicate or imply relative importance, or implicitly indicate a quantity of the indicated technical features. Therefore, a feature limited by “first” or “second” may explicitly or implicitly include at least one such feature. Furthermore, the technical solutions between the various examples may be combined with each other, but must be on the basis that the combination thereof can be implemented by a person of ordinary skill in the art. In case of a contradiction with the combination of the technical solutions or a failure to implement the combination, it should be considered that the combination of the technical solutions does not exist, and is not within the protection scope of the present application.

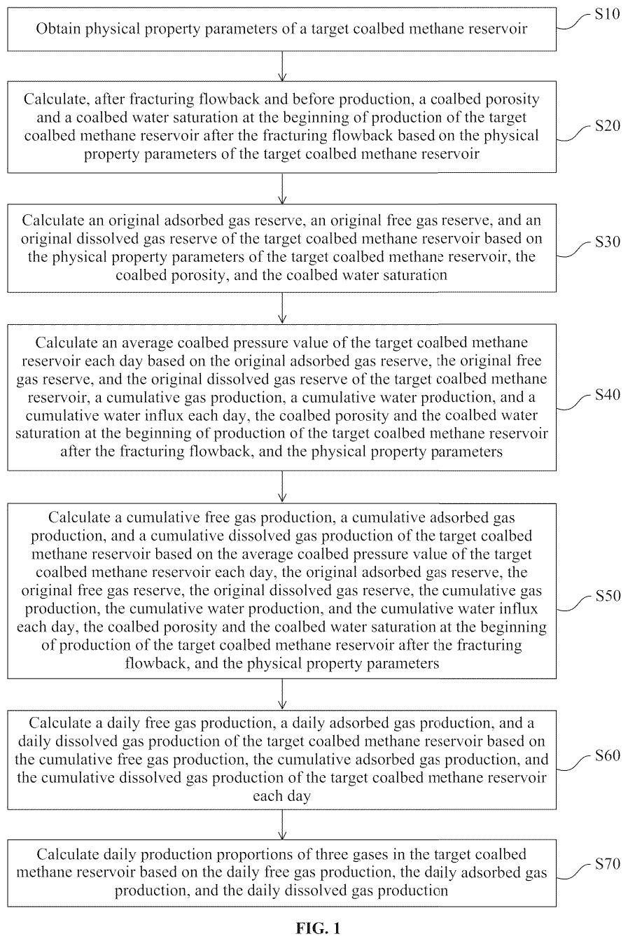

As shown in FIG. 1, an embodiment of the present application is to provide a method for evaluating production proportions of different gases in a deep coalbed methane reservoir, including the following steps.

In step S10, physical property parameters of a target coalbed methane reservoir are obtained.

The physical property parameters of the target coalbed methane reservoir include a coal rock density p_{c}, an initial coalbed pressure pᵢ, a coalbed temperature T, a coalbed controlled volume V, an initial coalbed porosity φᵢ, an initial coalbed water saturation S_{wi}, a pore compressibility Cₚ, an average pore diameter Da of coal matrix micropores, an average pore diameter D_{f} of coal matrix mesopores, a coal matrix surface wetting angle θ, a coal matrix shrinkage factor Cₐ, Langmuir volume V_{L} of adsorbed gas, Langmuir pressure p_{L} of the adsorbed gas, a relative gas density y_{g}, a volume factor of water B_{w}, an isothermal compressibility of water C_{w}, a dissolution factor of coalbed methane in water Cₛ, and a gas-water interfacial tension σ_{gw}.

Further, the coalbed controlled volume V may be calculated by the following formula (1):

$$V = A h = {L}_{H} L h \qquad ( 1 )$$

where V represents the coalbed controlled volume, 10⁶ m³; A represents a controlled area of the target coalbed methane reservoir, km²; h represents a thickness of the target coalbed methane reservoir, m; L_{H} represents a length of a horizontal well interval of the target coalbed methane reservoir, km; and L represents a spacing of horizontal wells in the target coalbed methane reservoir, km.

In step S20, after fracturing flowback and before production, a coalbed porosity and a coalbed water saturation at the beginning of production of the target coalbed methane reservoir after the fracturing flowback are calculated based on the physical property parameters of the target coalbed methane reservoir.

In step S30, an original adsorbed gas reserve, an original free gas reserve, and an original dissolved gas reserve of the target coalbed methane reservoir are calculated based on the physical property parameters of the target coalbed methane reservoir, the coalbed porosity, and the coalbed water saturation.

In step S40, an average coalbed pressure value of the target coalbed methane reservoir each day is calculated based on the original adsorbed gas reserve, the original free gas reserve, and the original dissolved gas reserve of the target coalbed methane reservoir, a cumulative gas production, a cumulative water production, and a cumulative water influx each day, the coalbed porosity and the coalbed water saturation at the beginning of production of the target coalbed methane reservoir after the fracturing flowback, and the physical property parameters.

In step S50, a cumulative free gas production, a cumulative adsorbed gas production, and a cumulative dissolved gas production of the target coalbed methane reservoir are calculated based on the average coalbed pressure value of the target coalbed methane reservoir each day, the original adsorbed gas reserve, the original free gas reserve, the original dissolved gas reserve, the cumulative gas production, the cumulative water production, and the cumulative water influx each day, the coalbed porosity and the coalbed water saturation at the beginning of production of the target coalbed methane reservoir after the fracturing flowback, and the physical property parameters.

In step S60, a daily free gas production, a daily adsorbed gas production, and a daily dissolved gas production of the target coalbed methane reservoir are calculated based on the cumulative free gas production, the cumulative adsorbed gas production, and the cumulative dissolved gas production of the target coalbed methane reservoir each day.

In step S70, daily production proportions of three gases in the target coalbed methane reservoir are calculated based on the daily free gas production, the daily adsorbed gas production, and the daily dissolved gas production.

Herein, based on the material balance principle for the deep coalbed methane reservoir and by considering the pressure difference between adsorbed gas pores and free gas pores and the effects of fracturing transformation on the physical property parameter changes of the coal reservoir and the dissolved gas, the cumulative free gas production, the cumulative adsorbed gas production, and the cumulative dissolved gas production of the target coalbed methane reservoir are calculated, and the daily free gas production, the daily adsorbed gas production, and the daily dissolved gas production are calculated based on the cumulative free gas production, the cumulative adsorbed gas production, and the cumulative dissolved gas production, thereby obtaining the production proportions of the three gases. The method for evaluating production proportions of adsorbed gas, free gas, and dissolved gas in a deep coalbed methane reservoir provided in the present application only requires known physical property parameters of the coal reservoir, fluid physical property parameters, and production performance data of the coalbed methane well, and enables the real-time calculation of the daily production proportions of gases of different types in the deep coalbed methane reservoir each day, thereby providing a reference for the analysis of coalbed methane well production performance.

In an embodiment of the present disclosure, the step of calculating, during the fracturing process, the coalbed porosity and the coalbed water saturation at the beginning of production of the target coalbed methane reservoir after the fracturing flowback based on the physical property parameters of the target coalbed methane reservoir includes:
obtaining a total fracturing fluid quantity during a fracturing process of the target coalbed methane reservoir and a cumulative flowback quantity during a flowback process;
calculating a net fracturing injection quantity based on the total fracturing fluid quantity and the cumulative flowback quantity;
calculating the coalbed porosity at the beginning of production of the target coalbed methane reservoir after the fracturing flowback based on a coalbed controlled volume, the net fracturing injection quantity, an initial porosity of the target coalbed methane reservoir, and a volume factor of water; and
calculating the coalbed water saturation based on the coalbed controlled volume, the initial porosity of the target coalbed methane reservoir, an initial water saturation, the net fracturing injection quantity, the volume factor of water, and the coalbed porosity at the beginning of production of the target coalbed methane reservoir after the fracturing flowback.

In this embodiment, in the target coalbed methane reservoir, the adsorbed gas primarily occurs within the micropores (with a pore diameter<2 nm) of the coal matrix, whereas the free gas, the dissolved gas, and water occur within cleat fractures (with a pore diameter>1000 nm), macropores (with a pore diameter between 50 nm and 1000 nm), and mesopores (with a pore diameter between 2 nm and 50 nm). The initial coalbed pressure pᵢ is essentially the initial fluid pressure within the cleat fractures, macropores, and mesopores of the coalbed, representing the initial pressure in the free pores. Since the matrix micropores do not contain a water phase, whereas the mesopores do, a capillary pressure exists between the matrix micropores and mesopores, which can be calculated by the following formula:

$${p}_{c} = 2 ( \frac{1}{{D}_{a}} - \frac{1}{{D}_{f}} ) {σ}_{g w} cos θ \qquad ( 2 )$$

where p_{c} represents the capillary pressure between the coalbed micropores and mesopores, MPa; Dₐ represents the average pore diameter of the coal matrix micropores, nm; D_{f} represents the average pore diameter of the coal matrix mesopores, nm; σ_{gw} represents the gas-water interfacial tension, mN/m; and θ represents the coal matrix surface wetting angle,°.

Within the known initial coalbed pressure pᵢ, the initial pressure pₘᵢ of the adsorbed gas in coalbed adsorption pores can be calculated by the following formula:

$${p}_{m i} = {p}_{i} + {p}_{c} \qquad ( 3 )$$

where pₘᵢ represents the initial pressure of the adsorbed gas in the coalbed adsorption pores, MPa; pᵢ represents the initial coalbed pressure, MPa; and p_{c} represents the capillary pressure between the coalbed micropores and mesopores, MPa.

The total fracturing fluid quantity W_{fi} during the fracturing process of the target coalbed methane reservoir and the cumulative flowback quantity W_{fp} during the flowback process are counted, and the net fracturing injection quantity W_{fin} is calculated by the following formula:

$${W}_{fin} = {W}_{fi} - {W}_{fp} \qquad ( 4 )$$

where W_{fin} represents the net fracturing injection quantity, 10⁶ m³; W_{fi} represents the total fracturing fluid quantity during the fracturing process of the target coalbed methane reservoir, 10⁶ m³; and W_{fp} represents the cumulative flowback quantity during the flowback process, 10⁶ m³.

The value of the coalbed controlled volume V, the initial coalbed porosity di, the net fracturing injection quantity W_{fin}, and the volume factor of water B_{w} are substituted into formula (5) for calculating the coalbed porosity φᵢₚ at the beginning of production of the target coalbed methane reservoir after the fracturing flowback:

$${ϕ}_{i p} = {ϕ}_{i} + \frac{{W}_{fin} {B}_{w}}{V} \qquad ( 5 )$$

where φᵢₚ represents the coalbed porosity at the beginning of production of the target coalbed methane reservoir after the fracturing flowback; φᵢ represents the initial coalbed porosity; W_{fin} represents the net fracturing injection quantity, 10⁶ m³; B_{w} represents the volume factor of water, m³/m³; and V represents the coalbed controlled volume, 10⁶ m³.

The value of the coalbed controlled volume V, the initial coalbed porosity φᵢ, the initial coalbed water saturation S_{wi}, the net fracturing injection quantity W_{fin}, the volume factor of water B_{w}, and the calculated coalbed porosity φᵢₚ at the beginning of production of the target coalbed methane reservoir after the fracturing flowback are substituted into formula (6) for calculating the coalbed water saturation S_{wip} at the beginning of production of the target coalbed methane reservoir after the fracturing flowback:

$${S}_{w i p} = \frac{{ϕ}_{i} {S}_{wi} + \frac{{W}_{fin} {B}_{w}}{V}}{{ϕ}_{ip}} \qquad ( 6 )$$

where S_{wip} represents the coalbed water saturation at the beginning of production of the target coalbed methane reservoir after the fracturing flowback; φᵢ represents the initial coalbed porosity; S_{wi} represents the initial coalbed water saturation; W_{fin} represents the net fracturing injection quantity, 10⁶ m³; B_{w} represents the volume factor of water, m³/m³; V represents the coalbed controlled volume, 10⁶ m³; and φᵢₚ represents the coalbed porosity at the beginning of production of the target coalbed methane reservoir after the fracturing flowback.

In an embodiment of the present application, the step of calculating the average coalbed pressure value of the target coalbed methane reservoir each day based on the original adsorbed gas reserve, the original free gas reserve, and the original dissolved gas reserve of the target coalbed methane reservoir, the cumulative gas production, the cumulative water production, and the cumulative water influx each day, the coalbed porosity and the coalbed water saturation at the beginning of production of the target coalbed methane reservoir after the fracturing flowback, and the physical property parameters includes:
establishing a material balance equation for adsorbed gas in adsorption pores according to the material balance principle for adsorbed gas desorption;
establishing a material balance equation for free gas and dissolved gas in free pores according to the material balance principle for the free gas and the dissolved gas;
establishing a material balance equation for the target coalbed methane reservoir according to the material balance equation for the adsorbed gas and the material balance equation for the free gas and the dissolved gas; and
substituting the original adsorbed gas reserve, the original free gas reserve, and the original dissolved gas reserve of the target coalbed methane reservoir, the cumulative gas production, the cumulative water production, the cumulative water influx, the coalbed porosity and the coalbed water saturation at the beginning of production of the target coalbed methane reservoir after the fracturing flowback, and the physical property parameters into the material balance equation for the target coalbed methane reservoir, and calculating the average coalbed pressure value by one of a Newton iteration method, a secant method, and a univariate cubic function calculation method.

In this embodiment, according to the material balance principle for adsorbed gas desorption, i.e., the cumulative desorption quantity of adsorbed gas in the target coalbed methane reservoir adsorption pores into free pores is equal to the original adsorbed gas reserve minus the current remaining adsorbed gas reserve, the material balance equation for adsorbed gas in adsorption pores may be established as follows:

$${G}_{p a} = V {ρ}_{c} \frac{{V}_{L} ( {p}_{i} + {p}_{c} )}{{p}_{L} + {p}_{i} + {p}_{c}} - V {ρ}_{c} \frac{{V}_{L} ( p + {p}_{c} )}{{p}_{L} + p + {p}_{c}} \qquad ( 7 )$$

where Gₚₐ represents a cumulative desorption quantity of adsorbed gas in the target coalbed methane reservoir adsorption pores into free pores, namely the cumulative adsorbed gas production, 10⁶ m³; ρ_{c} represents a coal rock density, t/m³; V_{L} represents Langmuir volume, m³/t; p_{L} represents Langmuir pressure, MPa; p_{c} represents a capillary pressure between coalbed micropores and mesopores, MPa; pᵢ represents an initial coalbed pressure, MPa; and p represents an average coal reservoir pressure, MPa.

During the production process of the target coalbed methane reservoir, coalbed methane is produced from the free pores by the coalbed methane well, while desorbed gas is supplied by the adsorption pores to the free pores. Therefore, for the free pores of the target coalbed methane reservoir, the cumulative gas production from the coalbed methane well is considered an outflow, whereas the cumulative desorption quantity from the adsorption pores is considered a supply. The net cumulative gas production from the free pores of the target coalbed methane reservoir (cumulative coalbed methane well production minus cumulative desorption quantity from the adsorption pores) is equal to the sum of the original free gas reserve and the original dissolved gas reserve in the free pores, minus the sum of the current free gas reserve and the current dissolved gas reserve in the free pores. Thus, the material balance equation for the free gas and the dissolved gas in the free pores of the target coalbed methane reservoir is established as follows:

$${G}_{p} - {G}_{p a} = \frac{V {ϕ}_{i p} ( 1 - {S}_{w i p} ) {Z}_{s c} {T}_{sc} {p}_{i}}{{p}_{s c} {Z}_{i} T} + V {ϕ}_{i p} {S}_{w i p} {C}_{s} {p}_{i} - \frac{V {ϕ}_{i p} {Z}_{s c} {T}_{s c} p}{{p}_{s c} T Z} [ 1 - {S}_{w i p} - ( {C}_{p} + {S}_{w i p} {C}_{w} ) ( {p}_{i} - p ) - \frac{{W}_{e} - {W}_{p} {B}_{w}}{V {ϕ}_{i p}} + {C}_{a} ( \frac{{p}_{i} + {p}_{c}}{{p}_{L} + {p}_{i} + {p}_{c}} - \frac{p + {p}_{c}}{{p}_{L} + {p}_{i} + {p}_{c}} ) ] - V {ϕ}_{i p} {C}_{s} p [ {S}_{w i p} + {S}_{w i p} {C}_{w} ( {p}_{i} - p ) + \frac{{W}_{e} - {W}_{p} {B}_{w}}{V {ϕ}_{i p}} ] \qquad ( 8 )$$

where Gₚ represents the cumulative gas production of the target coalbed methane reservoir, 10⁶ m³; Wₚ represents the cumulative water production of the target coalbed methane reservoir, 10⁶ m³; Wₑ represents the cumulative water influx during the production process, 10⁶ m³; φᵢₚ represents the coalbed porosity at the beginning of production of the target coalbed methane reservoir after the fracturing flowback; S_{wip} represents the coalbed water saturation at the beginning of production of the target coalbed methane reservoir after the fracturing flowback; p_{sc} represents the pressure under standard conditions, MPa, with a value of 0.101325; T_{sc} represents the temperature under standard conditions, K, with a value of 293.15; Z_{sc} represents the natural gas deviation factor under standard conditions, dimensionless, with a value of 1; pᵢ represents the initial coalbed pressure, MPa; T represents the coalbed temperature, K; Zᵢ represents the gas deviation factor under the initial coalbed pressure, dimensionless; p represents the average coal reservoir pressure, MPa; Z represents the gas deviation factor under the average coalbed pressure, dimensionless; Cₚ represents the pore volume compressibility, MPa⁻¹; C_{w} represents the isothermal compressibility of water, MPa⁻¹; Cₛ represents the dissolution factor of coalbed methane in water, MPa⁻¹; B_{w} represents the volume factor of water, m³/m³; and Cₐ represents the coal matrix shrinkage factor, dimensionless.

The formula (7) and the formula (8) may be added together, deriving the material balance equation for the target coalbed methane reservoir with consideration of the pressure difference between the adsorption pores and the free pores and the influence of the fracturing fluid, as shown in formula (9):

$${G}_{p} = V {ρ}_{c} \frac{{V}_{L} ( {p}_{i} + {p}_{c} )}{{p}_{L} + {p}_{i} + {p}_{c}} - V {ρ}_{c} \frac{{V}_{L} ( p + {p}_{c} )}{{p}_{L} + p + {p}_{c}} + \frac{V {ϕ}_{i p} ( 1 - {S}_{w i p} ) {Z}_{sc} {T}_{sc} {p}_{i}}{{p}_{sc} {Z}_{i} T} + V {ϕ}_{i p} {S}_{w i p} {C}_{s} {p}_{i} - \frac{V {ϕ}_{i p} {Z}_{s c} {T}_{s c} p}{{p}_{sc} T Z} [ 1 - {S}_{w i p} - ( {C}_{p} + {S}_{w i p} {C}_{w} ) ( {p}_{i} - p ) - \frac{{W}_{e} - {W}_{p} {B}_{w}}{V {ϕ}_{i p}} + {C}_{a} ( \frac{{p}_{i} + {p}_{c}}{{p}_{L} + {p}_{i} + {p}_{c}} - \frac{p + {p}_{c}}{{p}_{L} + p + {p}_{c}} ) ] - V {ϕ}_{i p} {C}_{s} p [ {S}_{w i p} + {S}_{w i p} {C}_{w} ( {p}_{i} - p ) + \frac{{W}_{e} - {W}_{p} {B}_{w}}{V {ϕ}_{i p}} ] \qquad ( 9 )$$

In this embodiment, the cumulative gas production Gₚ, the cumulative water production Wₚ, and the cumulative water influx Wₑ (typically with values of 0, unless the fracturing operation accidentally establishes hydraulic communication with the aquifer, leading to gradual water invasion into the coalbed during production as fluids are extracted) of the target coalbed methane reservoir each day, the coalbed porosity φᵢₚ at the beginning of production of the target coalbed methane reservoir after the fracturing flowback, the coalbed water saturation S_{wip} at the beginning of production of the target coalbed methane reservoir after the fracturing flowback, and the physical property parameters of the target coalbed methane reservoir are substituted into the formula (9) for ascertaining the average coalbed pressure value of the target coalbed methane reservoir each day by using a Newton iteration method or a secant method. During the solution process, the Dranchuk-Abou-Kassem method is applied to calculate the average gas deviation factor Z under any formation pressure based on the natural gas relative gravity γ_{g} and the reservoir temperature T.

As both the Newton iteration method and the secant method are implicit solution methods, they involve complex iterative loops and require the assistance of computer programs for implementation. To simplify the calculation, a straightforward explicit method for calculating the average reservoir pressure is proposed.

The expressions of the original adsorbed gas reserve, the original free gas reserve, and the original dissolved gas reserve in the formula (9) are denoted by Gₐᵢ, G_{fi}, and Gₛᵢ, respectively:

$$V {ρ}_{c} \frac{{V}_{L} ( {p}_{i} + {p}_{c} )}{{p}_{L} + {p}_{i} + {p}_{c}} = {G}_{a i} \qquad ( 11 )$$

$$\frac{V {ϕ}_{i p} ( 1 - {S}_{w i p} ) {Z}_{s c} {T}_{s c} {p}_{i}}{{p}_{s c} {Z}_{i} T} = {G}_{f i}$$

$$V {ϕ}_{i p} {S}_{w i p} {C}_{s} {p}_{i} = {G}_{s i}$$

Substituting the formula (11) into the formula (9) results in:

$${G}_{p} = {G}_{a i} + {G}_{f i} + {G}_{s i} - V {ρ}_{c} \frac{{V}_{L} ( p + {p}_{c} )}{{p}_{L} + p + {p}_{c}} - \frac{V {ϕ}_{i p} {Z}_{sc} {T}_{sc} p}{{p}_{sc} T Z} [ 1 - {S}_{w i p} - ( {C}_{p} + {S}_{w i p} {C}_{w} ) ( {p}_{i} - p ) - \frac{{W}_{e} - {W}_{p} {B}_{w}}{V {ϕ}_{i p}} + {C}_{a} ( \frac{{p}_{i} + {p}_{c}}{{p}_{L} + {p}_{i} + {p}_{c}} - \frac{p + {p}_{c}}{{p}_{L} + p + {p}_{c}} ) ] - V {ϕ}_{i p} {C}_{s} p [ {S}_{w i p} + {S}_{w i p} {C}_{w} ( {p}_{i} - p ) + \frac{{W}_{e} - {W}_{p} {B}_{w}}{V {ϕ}_{i p}} ] \qquad ( 12 )$$

Both sides of the formula (12) are multiplied by (p_{L}+p_{c}+p), and then organized into a univariate cubic equation of the average coalbed pressure p to derive:

$${p}^{3} + b {p}^{2} + c p + d = 0 \qquad ( 13 )$$

where the factor b is expressed as follows:

$$b = \frac{\begin{matrix}\frac{{Z}_{sc} {T}_{sc}}{{p}_{sc} ZT} [ 1 - {S}_{wip} - ( {C}_{p} + {S}_{wip} {C}_{w} ) \\ ( {p}_{i} - {p}_{L} - {p}_{c} ) + {C}_{a} ( \frac{{p}_{d} + {p}_{c}}{{p}_{L} + {p}_{d} + {p}_{c}} - 1 ) ]\end{matrix}}{[ \frac{{Z}_{sc} {T}_{sc}}{{p}_{sc} ZT} ( {C}_{p} + {S}_{wip} {C}_{w} ) - {C}_{s} {S}_{wip} {C}_{w} ]} + \frac{{C}_{s} [ {S}_{wip} + {S}_{wip} {C}_{w} ( {p}_{i} - {p}_{L} - {p}_{c} ) ]}{[ \frac{{Z}_{sc} {T}_{sc}}{{p}_{sc} ZT} ( {C}_{p} + {S}_{wip} {C}_{w} ) - {C}_{s} {S}_{wip} {C}_{w} ]} + \frac{( {C}_{s} - \frac{{Z}_{sc} {T}_{sc}}{{p}_{sc} ZT} ) \frac{( {W}_{e} - {W}_{p} {B}_{w} )}{V {ϕ}_{ip}}}{[ \frac{{Z}_{sc} {T}_{sc}}{{p}_{sc} ZT} ( {C}_{p} + {S}_{wip} {C}_{w} ) - {C}_{s} {S}_{wip} {C}_{w} ]} \qquad ( 14 )$$

where the factor c is expressed as follows:

$$C = \frac{\frac{( {G}_{p} - {G}_{ai} - {G}_{fi} - {G}_{si} )}{V {ϕ}_{ip}} + \frac{{V}_{L}}{{ϕ}_{ip}}}{[ \frac{{Z}_{sc} {T}_{sc}}{{p}_{sc} Z T} ( {C}_{p} + {S}_{wip} {C}_{w} ) - {C}_{s} {S}_{wip} {C}_{w} ]} + \frac{{C}_{s} ( {p}_{L} + {p}_{c} ) ( {S}_{wip} + {S}_{wip} {C}_{w} {p}_{i} )}{[ \frac{{Z}_{sc} {T}_{sc}}{{p}_{sc} Z T} ( {C}_{p} + {S}_{wip} {C}_{w} ) - {C}_{s} {S}_{wip} {C}_{w} ]} + \frac{\begin{matrix}\frac{{Z}_{sc} {T}_{sc}}{{p}_{sc} Z T} ( {p}_{L} + {p}_{c} ) [ 1 - {S}_{w i p} - ( {C}_{p} + \\ {S}_{w i p} {C}_{w} ) {p}_{i} + {C}_{a} ( \frac{{p}_{d} + {p}_{c}}{{p}_{L} + {p}_{d} + {p}_{c}} ) ]\end{matrix}}{[ \frac{{Z}_{sc} {T}_{sc}}{{p}_{sc} ZT} ( {C}_{p} + {S}_{w i p} {C}_{w} ) - {C}_{s} {S}_{w i p} {C}_{w} ]} + \frac{( {C}_{s} - \frac{{Z}_{sc} {T}_{sc}}{{p}_{sc} ZT} ) ( {p}_{L} + {p}_{c} ) \frac{( {W}_{e} - {W}_{p} {B}_{w} )}{V {ϕ}_{i p}}}{[ \frac{{Z}_{sc} {T}_{sc}}{{p}_{sc} ZT} ( {C}_{p} + {S}_{w i p} {C}_{w} ) - {C}_{s} {S}_{w i p} {C}_{w} ]} - \frac{\frac{{Z}_{sc} {T}_{sc}}{{p}_{sc} ZT} {C}_{a} {p}_{c}}{[ \frac{{Z}_{sc} {T}_{sc}}{{p}_{sc} ZT} ( {C}_{p} + {S}_{w i p} {C}_{w} ) - {C}_{s} {S}_{w i p} {C}_{w} ]} \qquad ( 15 )$$

where the factor dis expressed as follows:

$$d = \frac{\frac{( {G}_{p} - {G}_{ai} - {G}_{fi} - {G}_{si} )}{V {ϕ}_{ip}} ( {p}_{L} + {p}_{c} ) + \frac{{V}_{L}}{{ϕ}_{ip}} {p}_{c}}{[ \frac{{Z}_{sc} {T}_{sc}}{{p}_{sc} ZT} ( {C}_{p} + {S}_{wip} {C}_{w} ) - {C}_{S} {S}_{wip} {C}_{w} ]} \qquad ( 16 )$$

The formula (13) is organized into the form of y³+C₁y+C₂=0, and then y, C₁, and C₂ are respectively expressed as follows:

$$y = p + \frac{b}{3} \qquad ( 17 )$$

$${C}_{1} = \frac{3 c - {b}^{2}}{3} \qquad ( 18 )$$

$${C}_{2} = \frac{2 {b}^{3} - 9 b c + 27 d}{27} \qquad ( 19 )$$

One real solution to the equation y³+C₁y+C₂=0 is as follows:

$$y = {( - \frac{{c}_{2}}{2} + \sqrt{{( \frac{{c}_{2}}{2} )}^{2} + {( \frac{{c}_{1}}{3} )}^{3}} )}^{\frac{1}{3}} + {( - \frac{{c}_{2}}{2} - \sqrt{{( \frac{{c}_{2}}{2} )}^{2} + {( \frac{{c}_{1}}{3} )}^{3}} )}^{\frac{1}{3}} \qquad ( 20 )$$

Then, the average coalbed pressure p of the target coalbed methane reservoir each day may be obtained by transforming the formula (17), i.e., by formula (21):

$$p = y - \frac{b}{3} \qquad ( 21 )$$

When calculating the factors b, c, and d, the value of V may be given directly, or may be calculated by the formula (1); the values of Gₐᵢ, G_{fi}, and Gₛᵢ may be given directly based on preliminary understanding, or may be calculated by the formula (11); for the value of Z, the value of Zᵢ may be used as an initial value first, and after a new average coalbed pressure p is calculated, the new value of Z is calculated by the formula (10), and the value of Z in the previous step is then replaced with the calculated new value of Z; the value of p is calculated repeatedly and circularly; when the relative error between two calculations of p satisfies a relative criterion of 1×10⁻⁶, the circulation is stopped; and the last value of p calculated is used as the final value of the average coalbed pressure p.

In an embodiment of the present application, the cumulative free gas production of the target coalbed methane reservoir is calculated by a formula below:

$${G}_{p f} = {G}_{fi} - \frac{V {ϕ}_{ip} {Z}_{sc} {T}_{s c} p}{{p}_{sc} T Z} [ 1 - {S}_{w i p} - ( {C}_{p} + {S}_{w i p} {C}_{w} ) ( {p}_{i} - p ) - \frac{{W}_{e} - {W}_{p} {B}_{w}}{V {ϕ}_{ip}} + {C}_{a} ( \frac{{p}_{i} + {p}_{c}}{{p}_{L} + {p}_{i} + {p}_{c}} - \frac{p + {p}_{c}}{{p}_{L} + p + {p}_{c}} ) ] \qquad ( 22 )$$

where G_{pf} represents the cumulative free gas production of the target coalbed methane reservoir each day, 10⁶ m³.

In an embodiment of the present application, the cumulative dissolved gas production of the target coalbed methane reservoir is calculated by a formula below:

$${G}_{p s} = {G}_{s i} - V {ϕ}_{i p} {C}_{s} p [ {S}_{w i p} + {S}_{w i p} {C}_{w} ( {p}_{i} - p ) + \frac{{W}_{e} - {W}_{p} {B}_{w}}{V {ϕ}_{ip}} ] \qquad ( 23 )$$

where Gₚₛ represents the cumulative dissolved gas production of the target coalbed methane reservoir each day, 10⁶ m³.

In an embodiment of the present application, the cumulative adsorbed gas production of the target coalbed methane reservoir is calculated by a formula below:

$${G}_{p a} = V {ρ}_{c} \frac{{V}_{L} ( {p}_{i} + {p}_{c} )}{{p}_{L} + {p}_{i} + {p}_{c}} - V {ρ}_{c} \frac{{V}_{L} ( p + {p}_{c} )}{{p}_{L} + p + {p}_{c}}$$

where Gₚₐ represents a cumulative desorption quantity of adsorbed gas in the target coalbed methane reservoir into free pores, namely the cumulative adsorbed gas production, 10⁶ m³; ρ_{c} represents a coal rock density, t/m³; V_{L} represents Langmuir volume, m³/t; p_{L} represents Langmuir pressure, MPa; p_{c} represents a capillary pressure between target coalbed micropores and mesopores, MPa; pᵢ represents an initial coalbed pressure, MPa; and p represents an average coal reservoir pressure, MPa.

In some embodiments, if the cumulative free gas production of the current day is greater than the cumulative free gas production of the previous day, the daily free gas production of the current day is equal to the cumulative free gas production of the current day minus the cumulative free gas production of the previous day. If the cumulative free gas production of the current day is less than or equal to the cumulative free gas production of the previous day, the daily free gas production of the current day is 0. Therefore, the daily free gas production of the target coalbed methane reservoir is calculated by the following formula (24):

$${q}_{pf , j} = \{ \begin{matrix}{10}^{6} \frac{{G}_{pf , j} - {G}_{pf , j - 1}}{1} , & {G}_{pf , j} > {G}_{pf , j - 1} \\ 0 , & {G}_{pf , j} \leq {G}_{pf , j - 1}\end{matrix} \qquad ( 24 )$$

where q_{pf,j} represents the daily free gas production of the target coalbed methane reservoir on a current day (day j), m³/d; G_{pf,j} represents the cumulative free gas production of the target coalbed methane reservoir on the current day (day j), 10⁶ m³; and G_{pf,j-1} represents the cumulative free gas production of the target coalbed methane reservoir on a previous day (day j-1), 10⁶ m³.

In some embodiments, if the cumulative free gas production of the current day is greater than the cumulative free gas production of the previous day, the daily adsorbed gas production of the current day is equal to the cumulative adsorbed gas production of the current day minus the cumulative adsorbed gas production of the previous day. If the cumulative free gas production of the current day is less than or equal to the cumulative free gas production of the previous day, the daily adsorbed gas production of the current day is equal to the cumulative adsorbed gas production of the current day minus the cumulative adsorbed gas production of the previous day, plus the cumulative free gas production of the current day minus the cumulative free gas production of the previous day. Therefore, the daily adsorbed gas production may be calculated by the following formula (25):

$${q}_{p a , j} = \{ \begin{matrix}{10}^{6} \frac{{G}_{pa , j} - {G}_{pa , j - 1}}{1} , & {G}_{p f , j} > {G}_{p f , j - 1} \\ {10}^{6} \frac{{G}_{pa , j} - {G}_{pa , j - 1} + {G}_{pf , j} - {G}_{pf , j - 1}}{1} & {G}_{p f , j} \leq {G}_{p f , j - 1}\end{matrix} \qquad ( 25 )$$

where q_{pd,j} represents the daily adsorbed gas production of the target coalbed methane reservoir on a current day (day j), m³/d; G_{pa,j} represents the cumulative adsorbed gas production of the target coalbed methane reservoir on the current day (day j), 10⁶ m³; and G_{pa,j-1} represents the cumulative adsorbed gas production of the target coalbed methane reservoir on a previous day (day j-1), 10⁶ m³.

Further, the daily dissolved gas production of the current day may be calculated by subtracting the cumulative dissolved gas production of the previous day from the cumulative dissolved gas production of the current day. Therefore, the daily dissolved gas production may be calculated by the following formula (26):

$${q}_{p s , j} = 1 {0}^{6} \frac{{G}_{ps , j} - {G}_{ps , j - 1}}{1} \qquad ( 26 )$$

where q_{ps,j} represents the daily dissolved gas production of the target coalbed methane reservoir on the current day (day j), m³/d; G_{ps,j} represents the cumulative dissolved gas production of the target coalbed methane reservoir on the current day (day j), 10⁶ m³; and G_{ps,j-1} represents the cumulative dissolved gas production of the target coalbed methane reservoir on the previous day (day j-1), 10⁶ m³.

Further, the daily free gas production proportion f_{gpf} of the target coalbed methane reservoir on the current day (day j) is calculated by the formula (27):

$${f}_{qpf , j} = \frac{{q}_{pf , j}}{{q}_{pa , j} + {q}_{pf , j} + {q}_{ps , j}} \qquad ( 27 )$$

where f_{qpf,j} represents the daily free gas production proportion of a coalbed methane well on the current day (day j).

The daily adsorbed gas production proportion f_{qpa} of the target coalbed methane reservoir on the current day (day j) is calculated by the formula (28):

$${f}_{qpa , j} = \frac{{q}_{pa , j}}{{q}_{pa , j} + {q}_{pf , j} + {q}_{ps , j}} \qquad ( 28 )$$

where f_{qpa,j} represents the daily adsorbed gas production proportion of the coalbed methane well on the current day (day j).

The daily dissolved gas production proportion f_{qps} of the target coalbed methane reservoir on the current day (day j) is calculated by the formula (29):

$${f}_{qps , j} = \frac{{q}_{ps , j}}{{q}_{pa , j} + {q}_{pf , j} + {q}_{ps , j}} \qquad ( 29 )$$

where f_{qps,j} represents the daily dissolved gas production proportion of the coalbed methane well on the current day (day j).

Finally, to further illustrate the evaluation process of the method for evaluating production proportions of different gases in a deep coalbed methane reservoir as described in the present application, the following provides an example of the evaluation process for gas production proportions of a deep coal reservoir.

Firstly, the physical property parameters of the deep coal reservoir and the physical property parameters of the fluid are acquired, as shown in Table 1 below.

TABLE 1
Statistical Table of Physical Property Parameters of Fractured Deep Coal Reservoir

| Parameter | Value | Units |
|---|---|---|
| Coal rock density ρ_{c} | 1.5 | t/m³ |
| Initial coal reservoir pressure pᵢ | 21 | MPa |
| Coal reservoir temperature T | 343.15 | K |
| Deep coalbed methane reservoir controlled volume V | 3.507 | 10⁶ m³ |
| Initial coal reservoir porosity φᵢ | 0.07 | Decimals |
| Initial coal reservoir water saturation S_{wi} | 0.60433 | Decimals |
| Pore compressibility Cₚ | 0.005 | MPa⁻¹ |
| Average pore diameter Dₐ of coal matrix micropores | 2 | nm |
| Average pore diameter D_{f} of coal matrix mesopores | 10 | nm |
| Coal matrix surface wetting angle ϑ | 89 | ° |
| Coal matrix shrinkage factor Cₐ | 0.1 | Dimensionless |
| Langmuir volume V_{L} | 25 | m³/t |
| Langmuir pressure P_{L} | 3.21 | MPa |
| Relative gas density γ_{g} | 0.6 | Dimensionless |
| Gas deviation factor Zᵢ under initial coal reservoir pressure | 0.8895 | Dimensionless |
| Volume factor of water B_{w} | 1 | m³/m³ |
| Isothermal compressibility of water C_{w} | 0.000435 | MPa⁻¹ |
| Dissolution factor of coalbed methane in water Cₛ | 0.17 | MPa⁻¹ |
| Gas-water interfacial tension σ_{gw} | 60 | mN/m |

In the above table, the deep coalbed methane reservoir controlled volume V may be calculated by the formula (1). The thickness h=10 m of the deep coal reservoir, the length L_{H}=1.002 km of the horizontal well interval of the deep coalbed methane reservoir, and the spacing L=0.35 km of horizontal wells in the deep coalbed methane reservoir are substituted into the formula (1) to ascertain the deep coalbed methane reservoir controlled volume V=3.507 (10⁶ m³)

$$V = Ah = {L}_{H} Lh = 1.002 \times 0.35 \times 10 = 3.5 \; 07 ( {10}^{6} {m}^{3} )$$

Further, the capillary pressure between the matrix micropores and mesopores is calculated by the formula (2). The average pore diameter Dₐ=2 nm of the coal matrix micropores, the average pore diameter D_{f}=10 nm of the coal matrix mesopores, the gas-water interfacial tension σ_{gw}=60 mN/m, and the coal matrix surface wetting angle θ=89° are substituted into the formula (2) to ascertain the capillary pressure p_{c}=0.8377 (MPa) between the coal reservoir micropores and mesopores

$${p}_{c} = 2 ( \frac{1}{{D}_{a}} - \frac{1}{{D}_{f}} ) {σ}_{g w} cos θ = 2 ( \frac{1}{2} - \frac{1}{10} ) \times 60 \times cos ( 89 ° ) = 0.8377 ( MPa )$$

The initial coal reservoir pressure pᵢ=21 MPa and the capillary pressure between the coal reservoir micropores and mesopores p_{c}-0.8377 MPa are substituted into the formula (3) to ascertain the initial pressure of the adsorbed gas in the coal reservoir adsorption pores pₘᵢ=21.8377 (MPa).

$${p}_{m i} = {p}_{i} + {p}_{c} = 21 + 0.8377 = 21.8377 ( MPa )$$

The total fracturing fluid quantity W_{fi}=0.03 (10⁶ m³) during the fracturing process of the deep coalbed methane reservoir and the cumulative flowback quantity W_{fp}=0.0075 (10⁶ m³) during the flowback process are counted, and the net fracturing injection quantity W_{fin}=0.0225 (10⁶ m³) is calculated by the formula (4).

$${W}_{fin} = {W}_{fi} - {W}_{fp} = 0.03 - 0.0075 = 0.0 \; 225 ( {10}^{6} {m}^{3} )$$

The results are recorded in the following Table 2.

TABLE 2
Statistical Table of Fracturing and Flowback Data
of Deep Coalbed Methane Reservoir

| Parameter | Value | Units |
|---|---|---|
| Total fracturing fluid quantity W_{fi} | 0.03 | 10⁶ m³ |
| Cumulative flowback quantity W_{fp} | 0.0075 | 10⁶ m³ |
| Net fracturing injection quantity W_{fin} | 0.0225 | 10⁶ m³ |

The controlled volume V=3.507 (10⁶ m³) by the coalbed methane well, the initial coal reservoir porosity φᵢ=0.07, the net fracturing injection quantity W_{fin}=0.0225 (10⁶ m³), and the volume factor of water B_{w}=1 are substituted into the formula (5) to ascertain the coal reservoir porosity φᵢₚ=0.07642 at the beginning of production of the coalbed methane well after the fracturing flowback.

$${ϕ}_{i p} = {ϕ}_{i} + \frac{{W}_{fin} {B}_{w}}{V} = 0.07 + \frac{0.0225 \times 1}{3.507} = 0.07642$$

The controlled volume V=3.507 (10⁶ m³) by the coalbed methane well, the initial coal reservoir porosity φᵢ=0.07, the initial coal reservoir water saturation S_{wi}=0.60433, the net fracturing injection quantity W_{fin}=0.0225 (10⁶ m³), the volume factor of water B_{w}=1, and the calculated coal reservoir porosity φᵢₚ=0.07642 at the beginning of production of the coalbed methane well after the fracturing flowback are substituted into the formula (6) to ascertain the coal reservoir water saturation S_{wip}=0.63755 at the beginning of production of the coalbed methane well after the fracturing flowback.

$${S}_{w i p} = \frac{{ϕ}_{i} {S}_{w i} + \frac{{W}_{fin} {B}_{w}}{V}}{{ϕ}_{i p}} = \frac{0.07 \times 0.60433 + \frac{0.025 \times 1}{3.507}}{0.07642} = 0.63755$$

Further, the Dranchuk-Abou-Kassem method is applied to calculate the average gas deviation factor Z under any formation pressure based on the natural gas relative gravity γ_{g} and the reservoir temperature T, and the expression of Z is determined by fitting a univariate quartic polynomial of pressure is determined as follows:

$$Z = {A}_{4} {p}^{4} + {A}_{3} {p}^{3} + {A}_{2} {p}^{2} + {A}_{1} p + {A}_{0} = - 4.2357 \times 1 {0}^{- 7} {p}^{4} + 2.3073 \times 1 {0}^{- 5} {p}^{3} + 3.5607 \times 1 {0}^{- 5} {p}^{2} - 1.2258 \times 1 {0}^{- 2} p + 1$$

Thus, the quartic pressure factor A₄=−4.2357×10⁻⁷ MPa⁻⁴, the cubic pressure factor A₃=2.3073×10⁻⁵ MPa⁻³, the quadratic pressure factor A₂=3.5607×10⁻⁵ MPa⁻², the linear pressure factor A₁=−1.2258×10⁻² MPa⁻¹, and the constant term factor A₀=1 are determined.

The controlled volume V=3.507 (10⁶ m³) of the coalbed methane well, the coal reservoir porosity φᵢₚ=0.07642 at the beginning of production of the coalbed methane well after the fracturing flowback, the coal reservoir water saturation S_{wip}=0.63755 at the beginning of production of the coalbed methane well after the fracturing flowback, and the parameters in Table 1 are substituted into the formula (11) to ascertain the original adsorbed gas reserve Gₐᵢ=114.658463 (10⁶ m³), the original free gas reserve G_{fi}-19.334369 (10⁶ m³), and the original dissolved gas reserve Gₛᵢ=0.609958 (10⁶ m³).

$${G}_{a i} = V {ρ}_{c} \frac{{V}_{L} ( {p}_{i} + {p}_{c} )}{{p}_{L} + {p}_{i} + {p}_{c}} = 3.507 \times 1.5 \times \frac{25 \times ( 21 + 0.8377 )}{3.21 + 21 + 0.8377} = 144.658463 ( 1 {0}^{6} {m}^{3} )$$

$${G}_{f i} = \frac{V {ϕ}_{i p} ( 1 - {S}_{w i p} ) {Z}_{sc} {T}_{sc} {p}_{i}}{{p}_{s c} {Z}_{i} T} = \frac{3.507 \times 0.07642 \times ( 1 - 0.63755 ) \times 1 \times 293.15 \times 21}{0.101325 \times 0.8895 \times 343.15} = 19. 33 \; 4369 ( {10}^{6} {m}^{3} )$$

$${G}_{s i} = V {ϕ}_{i p} {S}_{w i p} {C}_{s} {p}_{i} = 3.507 \times 0.07642 \times 0.63755 \times 0.17 \times 21 = 0.609958 ( {10}^{6} {m}^{3} )$$

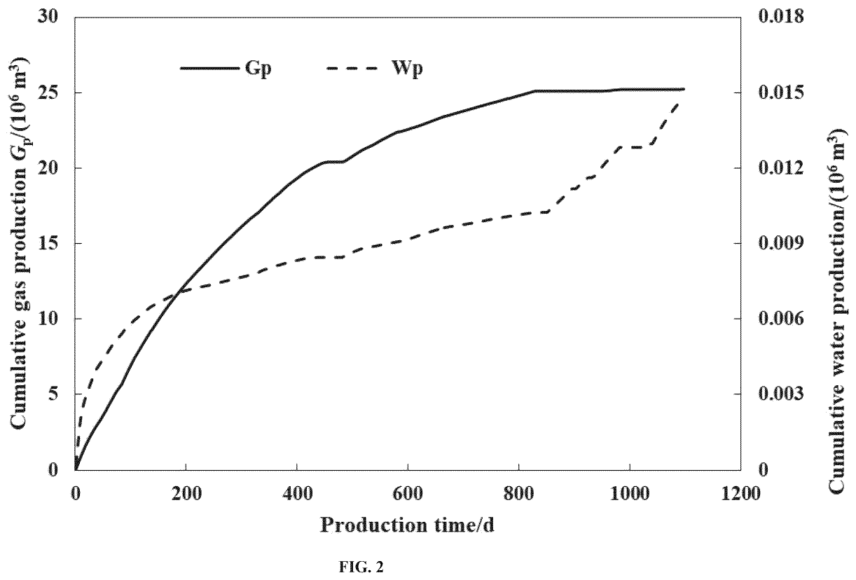

As can be seen from the above results, the curves of the cumulative gas production Gₚ and the cumulative water production Wₚ of the deep coalbed methane reservoir each day are as shown in FIG. 2.

Taking the production data of the deep coalbed methane reservoir on day 100 as an example, the cumulative gas production Gₚ=6.942087 (10⁶ m³), the cumulative water production Wₚ=0.00582697 (10⁶ m³), and the cumulative water influx Wₑ=0 on day 100, the coal reservoir porosity φᵢₚ=0.07642 at the beginning of production of the coalbed methane well after the fracturing flowback, the coal reservoir water saturation S_{wip}=0.63755 at the beginning of production of the coalbed methane well after the fracturing flowback, and the physical property parameters of the deep coal reservoir in Table 1 are substituted into the formulas (14), (15), and (16), with the initial value of Z being the value of Zᵢ, i.e., 0.8895, to ascertain:
the factor b as:

$$b = \frac{\begin{matrix}\frac{{Z}_{sc} {T}_{sc}}{{p}_{sc} ZT} [ 1 - {S}_{wip} - ( {C}_{p} + {S}_{wip} {C}_{w} ) ( {p}_{i} - {p}_{L} - {p}_{c} ) + \\ {C}_{a} ( \frac{{p}_{d} + {p}_{c}}{{p}_{L} + {p}_{d} + {p}_{c}} - 1 ) ]\end{matrix}}{[ \frac{{Z}_{sc} {T}_{sc}}{{p}_{sc} ZT} ( {C}_{p} + {S}_{wip} {C}_{w} ) - {C}_{s} {S}_{wip} {C}_{w} ]} + \frac{{C}_{s} [ {S}_{wip} + {S}_{wip} {C}_{w} ( {p}_{i} - {p}_{L} - {p}_{c} ) ]}{[ \frac{{Z}_{sc} {T}_{sc}}{{p}_{sc} ZT} ( {C}_{p} + {S}_{wip} {C}_{w} ) - {C}_{s} {S}_{wip} {C}_{w} ]} + \frac{( {C}_{s} - \frac{{Z}_{sc} {T}_{sc}}{{p}_{sc} ZT} ) \frac{( {W}_{e} - {W}_{p} {B}_{w} )}{V {ϕ}_{ip}}}{[ \frac{{Z}_{sc} {T}_{sc}}{{p}_{sc} ZT} ( {C}_{p} + {S}_{wip} {C}_{w} ) - {C}_{s} {S}_{wip} {C}_{w} ]} = 55.58135 ( MPa )$$

the factor c as:

$$c = \frac{\frac{( {G}_{p} - {G}_{ai} - {G}_{fi} - {G}_{si} )}{V {ϕ}_{ip}} + \frac{{V}_{L}}{{ϕ}_{ip}}}{[ \frac{{Z}_{sc} {T}_{sc}}{{p}_{sc} ZT} ( {C}_{p} + {S}_{wip} {C}_{w} ) - {C}_{s} {S}_{wip} {C}_{w} ]} + \frac{{C}_{s} ( {p}_{L} - {p}_{c} ) ( {S}_{wip} + {S}_{wip} {C}_{w} {p}_{i} )}{[ \frac{{Z}_{sc} {T}_{sc}}{{p}_{sc} ZT} ( {C}_{p} + {S}_{wip} {C}_{w} ) - {C}_{s} {S}_{wip} {C}_{w} ]} + \frac{\frac{{Z}_{sc} {T}_{sc}}{{p}_{sc} ZT} ( {p}_{L} + {p}_{c} ) [ 1 - {S}_{wip} - ( {C}_{p} + {S}_{wip} {C}_{w} ) {p}_{i} + {C}_{a} ( \frac{{p}_{d} + {p}_{c}}{{p}_{L} + {p}_{d} + {p}_{c}} ) ]}{[ \frac{{Z}_{sc} {T}_{sc}}{{p}_{sc} ZT} ( {C}_{p} + {S}_{wip} {C}_{w} ) - {C}_{s} {S}_{wip} {C}_{w} ]} + \frac{( {C}_{s} - \frac{{Z}_{sc} {T}_{sc}}{{p}_{sc} ZT} ) ( {p}_{L} + {p}_{c} ) \frac{( {W}_{e} - {W}_{p} {B}_{w} )}{V {ϕ}_{ip}}}{[ \frac{{Z}_{sc} {T}_{sc}}{{p}_{sc} ZT} ( {C}_{p} + {S}_{wip} {C}_{w} ) - {C}_{s} {S}_{wip} {C}_{w} ]} - \frac{\frac{{Z}_{sc} {T}_{sc}}{{p}_{sc} ZT} {C}_{a} {p}_{c}}{[ \frac{{Z}_{sc} {T}_{sc}}{{p}_{sc} ZT} ( {C}_{p} + {S}_{wip} {C}_{w} ) - {C}_{s} {S}_{wip} {C}_{w} ]} = 557.0824674 ( {MPa}^{2} )$$

the factor d as:

$$d = \frac{\frac{( {G}_{p} - {G}_{a i} - {G}_{fi} - {G}_{s i} )}{V {ϕ}_{i p}} ( {p}_{L} + {p}_{c} ) + \frac{{V}_{L}}{{ϕ}_{i p}} {p}_{c}}{[ \frac{{Z}_{s c} {T}_{s c}}{{p}_{s c} Z T} ( {C}_{p} + {S}_{wip} {C}_{w} ) - {C}_{s} {S}_{wip} {C}_{w} ]} = - 3 357.3 ( {MPa}^{3} )$$

The values of C₁ and C₂ are respectively calculated by the formula (18) and the formula (19) as follows:

$${C}_{1} = \frac{3 c - {b}^{2}}{3} = - 472.68$$

$${C}_{2} = \frac{2 {b}^{3} - 9 b c + 27 d}{27} = - 27959.4$$

The value of y is calculated by the formula (20) as follows:

$$y = {( - \frac{{C}_{2}}{2} + \sqrt{{( \frac{{C}_{2}}{2} )}^{2} + {( \frac{{C}_{1}}{3} )}^{3}} )}^{\frac{1}{3}} + {( - \frac{{C}_{2}}{2} - \sqrt{{( \frac{{C}_{2}}{2} )}^{2} + {( \frac{{C}_{1}}{3} )}^{3}} )}^{\frac{1}{3}} = 35.5002$$

The average coal reservoir pressure p of the deep coalbed methane reservoir on day 100 is calculated by the formula (21) as follows:

$$p = y - \frac{b}{3} = 35.5002 - \frac{55.58135}{3} = 16.97308 ( MPa )$$

The calculated average coal reservoir pressure p=16.97308 (MPa) on day 100 is substituted into the formula (10) for calculating the new value of Z:

$$Z = {A}_{4} {p}^{4} + {A}_{3} {p}^{3} + {A}_{2} {p}^{2} + {A}_{1} p + {A}_{0} = - 4.2357 \times 1 {0}^{- 7} {p}^{4} + 2.3073 \times 1 {0}^{- 5} {p}^{3} + 3.5607 \times 1 {0}^{- 5} {p}^{2} - 1.2258 \times 1 {0}^{- 2} p + 1 = - 4.2357 \times 1 {0}^{- 7} \times 16.9730 {8}^{4} + 2.3073 \times 1 {0}^{- 5} \times 16.9730 {8}^{3} + 3.5607 \times 1 {0}^{- 5} \times 16.9730 {8}^{2} - 1.2258 \times 1 {0}^{- 2} \times 16.97308 + 1 = 0.87986839$$

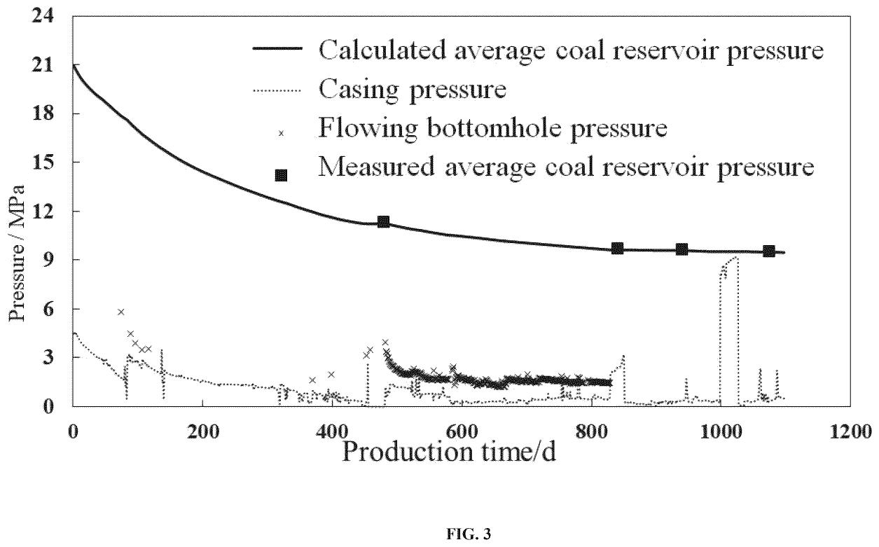

The value of Z in the previous step is then replaced with the calculated new value of Z. The value of p is calculated repeatedly and circularly. It has been found that, when the relative error of two calculations of p after 3 circulations satisfies a relative criterion of 1×10⁻⁶, the circulation is stopped, and the last value of p=16.89144 (MPa) calculated is used as the final value of the average coal reservoir pressure p on day 100. The average coal reservoir pressure p each day and the measured casing pressure as well as the flowing bottomhole pressure are plotted in the same graph, finally forming the curves as shown in FIG. 3.

Taking the data of the deep coalbed methane reservoir on day 100 as an example, the calculated average coal reservoir pressure p=16.89144 (MPa) of the deep coalbed methane reservoir on day 100, the original free gas reserve G_{fi}=19.334369 (10⁶ m³) controlled by the coalbed methane gas, the cumulative gas production Gₚ=6.942087 (10⁶ m³), the cumulative water production Wₚ=0.00582697 (10⁶ m³), and the cumulative water influx Wₑ=0 on day 100, the coal reservoir porosity φᵢₚ=0.07642 at the beginning of production of the coalbed methane well after the fracturing flowback, the coal reservoir water saturation S_{wip}=0.63755 at the beginning of production of the coalbed methane well after the fracturing flowback, and part of the desired parameters in Table 1 are substituted into the formula (22) to ascertain the cumulative free gas production G_{pf}=3.499893 (10⁶ m³) of the deep coalbed methane reservoir on day 100.

$${G}_{p f} = {G}_{f i} - \frac{V {ϕ}_{i p} {Z}_{s c} {T}_{s c} p}{{p}_{s c} T Z} [ 1 - {S}_{w i p} - ( {C}_{p} + {S}_{w i p} {C}_{w} ) ( {p}_{i} - p ) - \frac{{W}_{e} - {W}_{p} {B}_{w}}{V {ϕ}_{i p}} + {C}_{a} ( \frac{{p}_{i} + {p}_{c}}{{p}_{L} + {p}_{i} + {p}_{c}} - \frac{p + {p}_{c}}{{p}_{L} + p + {p}_{c}} ) ] = 3.4 \; 99893 ( {10}^{6} {m}^{3} )$$

The calculated average coal reservoir pressure p=16.89144 (MPa) of the deep coalbed methane reservoir on day 100 and part of the desired parameters in Table 1 are substituted into the formula (7) to ascertain the cumulative adsorbed gas production Gₚₐ=3.307003 (10⁶ m³) of the deep coalbed methane reservoir on day 100.

$${G}_{p a} = V {ρ}_{c} \frac{{V}_{L} ( {p}_{i} + {p}_{c} )}{{p}_{L} + {p}_{i} + {p}_{c}} - V {ρ}_{c} \frac{{V}_{L} ( p + {p}_{c} )}{{p}_{L} + p + {p}_{c}} = 3.507 \times 1.5 \times \frac{25 \times ( 21 + 0.8377 )}{3.21 + 21 + 0.8377} - 3.507 \times 1.5 \times \frac{25 \times ( 16.89144 + 0.8377 )}{3.21 + 16.89144 + 0.8377} = 3.3 \; 07003 ( {10}^{6} {m}^{3} )$$

The calculated average coal reservoir pressure p=16.89144 (MPa) of the deep coalbed methane reservoir on day 100, the original dissolved gas reserve Gₛᵢ=0.609958 (10⁶ m³) controlled by the coalbed methane well, the cumulative gas production Gₚ=6.942087 (10⁶ m³), the cumulative water production Wₚ=0.00582697 (10⁶ m³), and the cumulative water influx Wₑ=0 on day 100, the coal reservoir porosity φᵢₚ=0.07642 at the beginning of production of the coalbed methane well after the fracturing flowback, the coal reservoir water saturation S_{wip}=0.63755 at the beginning of production of the coalbed methane well after the fracturing flowback, and part of the desired parameters in Table 1 are substituted into the formula (23) to ascertain the cumulative dissolved gas production Gₚₛ=0.119336 (10⁶ m³) of the deep coalbed methane reservoir on day 100.

$${G}_{p s} = {G}_{s i} - V {ϕ}_{i p} {C}_{s} p [ {S}_{w i p} + {S}_{w i p} {C}_{w} ( {p}_{i} - p ) + \frac{{W}_{e} - {W}_{p} {B}_{w}}{V {ϕ}_{i p}} ] = 0.1 \; 19336 ( {10}^{6} {m}^{3} )$$

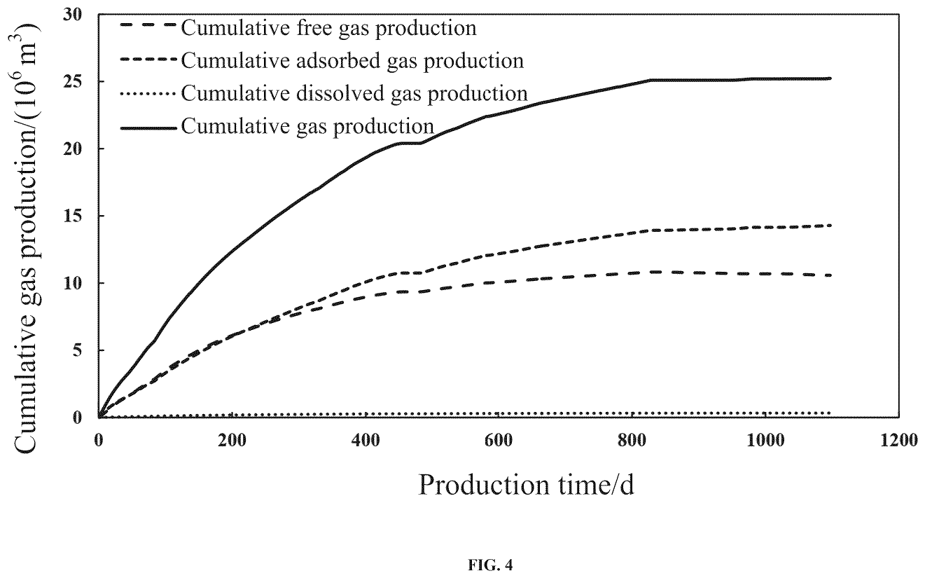

In the same way, the cumulative adsorbed gas production Gₚₐ, the cumulative free gas production G_{pf}, and the cumulative dissolved gas production Gₚₛ of the deep coalbed methane reservoir each day are calculated, and plotted, together with the total cumulative gas production, in the same graph, as shown in FIG. 4.

Taking the data of the deep coalbed methane reservoir on day 100 and day 99 as an example, the daily free gas production, the daily adsorbed gas production, and the daily dissolved gas production of the deep coalbed methane reservoir on day 100 are calculated.

Since the cumulative free gas production 3.499893 (10⁶ m³) on day 100 is greater than the cumulative free gas production 3.463176 (10⁶ m³) on day 99, the daily free gas production on day 100 is calculated by the formula (24) as q_{pf,100}=36717 (m³/d).

$${q}_{p f , 100} = 1 {0}^{6} \frac{{G}_{pf , 100} - {G}_{p f , 99}}{1} = 1 {0}^{6} \frac{3.499893 - 3.463176}{1} = 36717 ( {m}^{3} / d )$$

Since the cumulative free gas production 3.499893 (10⁶ m³) on day 100 is greater than the cumulative free gas production 3.463176 (10⁶ m³) on day 99, the cumulative adsorbed gas production on day 100 and the cumulative adsorbed gas production on day 99 are substituted into the formula (25) to calculate the daily adsorbed gas production on day 100 as q_{pa,100}=33855 (m³/d).

$${q}_{p a , 100} = 1 {0}^{6} \frac{{G}_{p a , 100} - {G}_{pa , 99}}{1} = 1 {0}^{6} \times \frac{3.307003 - 3.273148}{1} = 33855 ( {m}^{3} / d )$$

The cumulative dissolved gas production on day 100 and the cumulative dissolved gas production on day 99 are substituted into the formula (26) to calculate the daily dissolved gas production on day 100 as q_{ps,100}=1023 (m³/d).

$${q}_{p s , 100} = 1 {0}^{6} \frac{{G}_{ps , 100} - {G}_{p s , 99}}{1} = 1 {0}^{6} \times \frac{0.119336 - 0.118313}{1} = 1023 ( {m}^{3} / d )$$

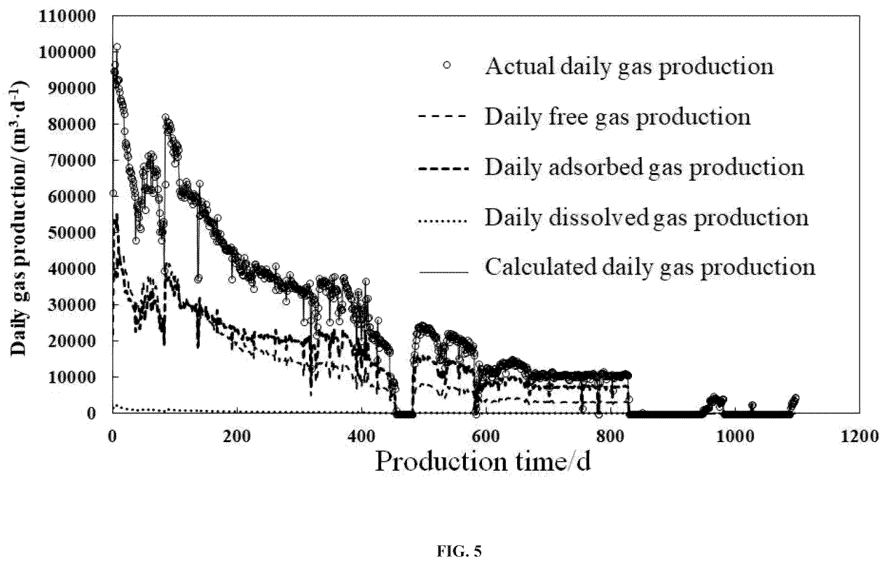

In the same way, the daily adsorbed gas production q_{pa,j}, the daily free gas production q_{pf,j}, and the daily dissolved gas production q_{ps,j} of the deep coalbed methane reservoir each day are calculated, and plotted, together with the daily gas production of the coalbed methane well, in the same graph, as shown in FIG. 5.

Taking the data of the deep coalbed methane reservoir on day 100 as an example, the daily free gas production proportion in the deep coalbed methane reservoir on day 100 is calculated by the formula (27) as f_{qpf}=51.28%.

$${f}_{qpf , 100} = \frac{{q}_{pf , 100}}{{q}_{p a , 100} + {q}_{pf , 100} + {q}_{p s , 100}} = \frac{36717}{33855 + 36717 + 1023} = 51.28 \%$$

The daily adsorbed gas production proportion in the deep coalbed methane reservoir on day 100 is calculated by the formula (28) as f_{qpa}=47.29%.

$${f}_{qpa , j 100} = \frac{{q}_{pa , 100}}{{q}_{pa , 100} + {q}_{pf , 100} + {q}_{p s , 100}} = \frac{33855}{33855 + 36717 + 1023} = 47.29 \%$$

The daily dissolved gas production proportion in the deep coalbed methane reservoir on day 100 is calculated by the formula (29) as f_{qps}=1.43%.

$${f}_{qps , 100} = \frac{{q}_{p s , 100}}{{q}_{pa , 100} + {q}_{pf , 100} + {q}_{p s , 100}} = \frac{1023}{33855 + 36717 + 1023} = 1.43 \%$$

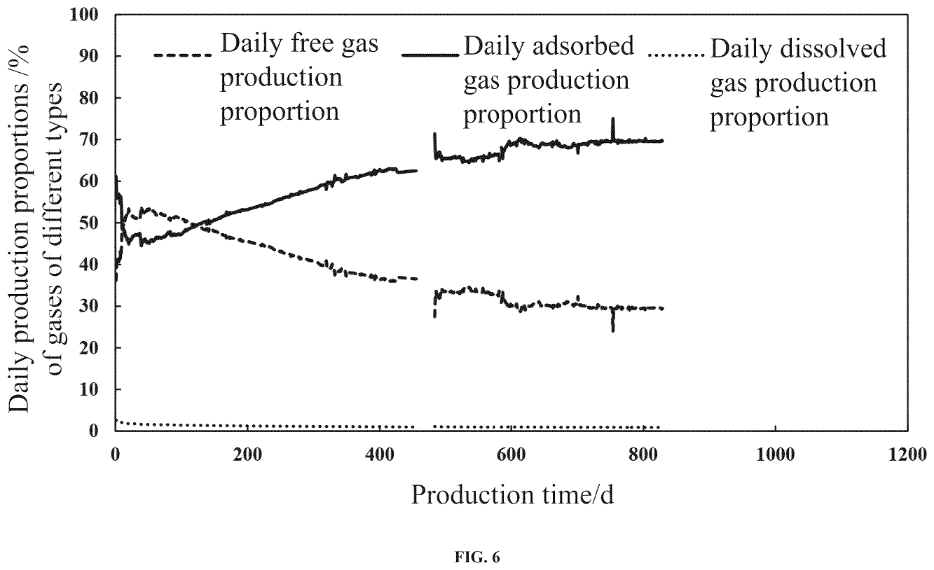

In the same way, the daily free gas production proportion f_{qpf,j}, the daily adsorbed gas production proportion f_{qpa,j}, and the daily dissolved gas production proportion f_{qps,j} each day in the deep coalbed methane reservoir are calculated, and plotted in the same graph, as shown in FIG. 6.

A second aspect of the present application provides a device for evaluating production proportions of different gases in a deep coalbed methane reservoir, including:
a memory configured to store instructions; and
a processor configured to invoke the instructions from the memory and implement the method for evaluating production proportions of different gases in a deep coalbed methane reservoir described above when executing the instructions.

The memory may include a non-persistent memory, a random access memory (RAM) and/or a non-volatile memory in a computer-readable medium, such as a read-only memory (ROM) or a flash RAM. The memory is an example of the computer-readable medium.

The computer-readable medium includes both persistent and non-persistent and removable and non-removable media, and storage of information may be implemented by any method or technology. The information may be computer-readable instructions, data structures, modules of programs, or other data. Examples of the storage medium of the computer include but are not limited to a phase-change RAM (PRAM), a static random access memory (SRAM), a dynamic random access memory (DRAM), other types of RAMs and ROMs, an electrically erasable programmable read-only memory (EEPROM), a flash memory or other memory techniques, a compact disk read-only memory (CD-ROM), a digital versatile disc (DVD), or other optical storage, a cassette type magnetic tape, disk storage, or other magnetic storage device, or any other non-transmission medium, that can be used to store information accessible by computing devices. The computer-readable medium, as defined herein, excludes computer-readable transitory media, such as modulated data signals and carrier waves.

It should also be noted that the term “comprise”, “include”, or any other variant thereof is intended to encompass a non-exclusive inclusion, such that a process, method, product, or device that includes a series of elements includes not only those elements, but also other elements not explicitly listed, or elements that are inherent to such a process, method, product, or device. Without more restrictions, an element defined by the phrase “including a . . . ” does not exclude the presence of another same element in a process, method, product, or device that includes the element.

The foregoing is merely illustrative of the embodiments of the present application and is not intended to limit the present application. Various changes and modifications can be made to the present application by those skilled in the art. Any modifications, equivalent replacements, improvements, etc. made within the spirit and principle of the present application should be included within the protection scope of the claims of the present application.

What is claimed is:

1. A method for evaluating production proportions of different gases in a deep coalbed methane reservoir, comprising following steps:
obtaining physical property parameters of a target coalbed methane reservoir;
calculating, after fracturing flowback and before production, a coalbed porosity and a coalbed water saturation at the beginning of production of the target coalbed methane reservoir after the fracturing flowback based on the physical property parameters of the target coalbed methane reservoir;
calculating an original adsorbed gas reserve, an original free gas reserve, and an original dissolved gas reserve of the target coalbed methane reservoir based on the physical property parameters of the target coalbed methane reservoir, the coalbed porosity, and the coalbed water saturation;
calculating an average coalbed pressure value of the target coalbed methane reservoir each day based on the original adsorbed gas reserve, the original free gas reserve, and the original dissolved gas reserve of the target coalbed methane reservoir, a cumulative gas production, a cumulative water production, and a cumulative water influx each day, the coalbed porosity and the coalbed water saturation at the beginning of production of the target coalbed methane reservoir after the fracturing flowback, and the physical property parameters;
calculating a cumulative free gas production, a cumulative adsorbed gas production, and a cumulative dissolved gas production of the target coalbed methane reservoir based on the average coalbed pressure value of the target coalbed methane reservoir each day, the original adsorbed gas reserve, the original free gas reserve, the original dissolved gas reserve, the cumulative gas production, the cumulative water production, and the cumulative water influx each day, the coalbed porosity and the coalbed water saturation at the beginning of production of the target coalbed methane reservoir after the fracturing flowback, and the physical property parameters;
calculating a daily free gas production, a daily adsorbed gas production, and a daily dissolved gas production of the target coalbed methane reservoir based on the cumulative free gas production, the cumulative adsorbed gas production, and the cumulative dissolved gas production of the target coalbed methane reservoir each day; and
calculating daily production proportions of free gas, adsorbed gas, and dissolved gas in the target coalbed methane reservoir based on the daily free gas production, the daily adsorbed gas production, and the daily dissolved gas production.

2. The method according to claim 1, wherein the step of calculating, after fracturing flowback and before production, a coalbed porosity and a coalbed water saturation at the beginning of production of the target coalbed methane reservoir after the fracturing flowback based on the physical property parameters of the target coalbed methane reservoir comprises:
obtaining a total fracturing fluid quantity during a fracturing process of the target coalbed methane reservoir and a cumulative flowback quantity during a flowback process;
calculating a net fracturing injection quantity based on the total fracturing fluid quantity and the cumulative flowback quantity;
calculating the coalbed porosity at the beginning of production of the target coalbed methane reservoir after the fracturing flowback based on a coalbed controlled volume, the net fracturing injection quantity, an initial porosity of the target coalbed methane reservoir, and a volume factor of water; and
calculating the coalbed water saturation based on the coalbed controlled volume, the initial porosity of the target coalbed methane reservoir, an initial water saturation, the net fracturing injection quantity, the volume factor of water, and the coalbed porosity at the beginning of production of the target coalbed methane reservoir after the fracturing flowback.

3. The method according to claim 1, wherein the step of calculating an average coalbed pressure value of the target coalbed methane reservoir each day based on the original adsorbed gas reserve, the original free gas reserve, and the original dissolved gas reserve of the target coalbed methane reservoir, a cumulative gas production, a cumulative water production, and a cumulative water influx each day, the coalbed porosity and the coalbed water saturation at the beginning of production of the target coalbed methane reservoir after the fracturing flowback, and the physical property parameters comprises:
establishing a material balance equation for adsorbed gas in adsorption pores according to the material balance principle for adsorbed gas desorption;
establishing a material balance equation for free gas and dissolved gas in free pores according to the material balance principle for the free gas and the dissolved gas;
establishing a material balance equation for the target coalbed methane reservoir according to the material balance equation for the adsorbed gas and the material balance equation for the free gas and the dissolved gas; and
substituting the original adsorbed gas reserve, the original free gas reserve, and the original dissolved gas reserve of the target coalbed methane reservoir, the cumulative gas production, the cumulative water production, the cumulative water influx, the coalbed porosity and the coalbed water saturation at the beginning of production of the target coalbed methane reservoir after the fracturing flowback, and the physical property parameters into the material balance equation for the target coalbed methane reservoir, and calculating the average coalbed pressure value by one of a Newton iteration method, a secant method, and a univariate cubic function calculation method.

4. The method according to claim 3, wherein the calculating the average coalbed pressure value by a univariate cubic function calculation method comprises:
establishing a univariate cubic function model according to the material balance equation for the target coalbed methane reservoir;
calculating a quadratic factor, a linear factor, and a constant term of the univariate cubic function model based on the original adsorbed gas reserve, the original free gas reserve, and the original dissolved gas reserve of the target coalbed methane reservoir, the cumulative gas production, the cumulative water production, and the cumulative water influx each day, the coalbed porosity and the coalbed water saturation at the beginning of production of the target coalbed methane reservoir after the fracturing flowback, and the physical property parameters; and
calculating the average coalbed pressure value according to the quadratic factor, the linear factor, and the constant term of the univariate cubic function model.

5. The method according to claim 1, wherein the cumulative free gas production of the target coalbed methane reservoir is calculated by a formula below:
$${G}_{pf} = {G}_{fi} - \frac{V {ϕ}_{i p} {Z}_{s c} {T}_{s c} p}{{p}_{s c} T Z} [ 1 - {S}_{w i p} - ( {C}_{p} + {S}_{w i p} {C}_{w} ) ( {p}_{i} - p ) - \frac{{W}_{e} - {W}_{p} {B}_{w}}{V {ϕ}_{i p}} + {C}_{a} ( \frac{{p}_{i} + {p}_{c}}{{p}_{L} + {p}_{i} + {p}_{c}} - \frac{p + {p}_{c}}{{p}_{L} + p + {p}_{c}} ) ]$$
wherein G_{pf} represents the cumulative free gas production of the target coalbed methane reservoir each day, 10⁶ m³; G_{fi} represents an original free gas reserve controlled by the target coalbed methane reservoir; Wₚ represents a cumulative water production of the target coalbed methane reservoir, 10⁶ m³; Wₑ represents a cumulative water influx during a production process, 10⁶ m³; φᵢₚ represents the coalbed porosity at the beginning of production of the target coalbed methane reservoir after the fracturing flowback; V represents a coalbed controlled volume, 10⁶ m³; S_{wip} represents the coalbed water saturation at the beginning of production of the target coalbed methane reservoir after the fracturing flowback; p_{sc} represents a pressure under standard conditions, MPa, with a value of 0.101325; T_{sc} represents a temperature under standard conditions, K, with a value of 293.15; Z_{sc} represents a natural gas deviation factor under standard conditions, dimensionless, with a value of 1; T represents a target coalbed temperature, K; Zᵢ represents a gas deviation factor under an initial coalbed pressure, dimensionless; p_{L} represents Langmuir pressure, MPa; p_{c} represents a capillary pressure between coalbed micropores and mesopores, MPa; pᵢ represents the initial coalbed pressure, MPa; p represents an average coal reservoir pressure, MPa; Z represents a gas deviation factor under an average coalbed pressure, dimensionless; Cₚ represents a pore volume compressibility, MPa⁻¹; C_{w} represents an isothermal compressibility of water, MPa⁻¹; Cₛ represents a dissolution factor of coalbed methane in water, MPa⁻¹; B_{w} represents a volume factor of water, m³/m³; and Cₐ represents a coal matrix shrinkage factor, dimensionless.

6. The method according to claim 1, wherein the cumulative dissolved gas production of the target coalbed methane reservoir is calculated by a formula below:
$${G}_{p s} = {G}_{s i} - V {ϕ}_{i p} {C}_{S} p [ {S}_{w i p} + {S}_{w i p} {C}_{w} ( {p}_{i} - p ) + \frac{{W}_{e} - {W}_{p} {B}_{w}}{V {ϕ}_{i p}} ]$$
wherein Gₚₛ represents the cumulative dissolved gas production of the target coalbed methane reservoir each day, 10⁶ m³; Gₛᵢ represents an original dissolved gas reserve controlled by the target coalbed methane reservoir; Wₚ represents a cumulative water production of the target coalbed methane reservoir, 10⁶ m³; Wₑ represents a cumulative water influx during a production process, 10⁶ m³; φᵢₚ represents the coalbed porosity at the beginning of production of the target coalbed methane reservoir after the fracturing flowback; V represents a coalbed controlled volume, 10⁶ m³; S_{wip} represents the coalbed water saturation at the beginning of production of the target coalbed methane reservoir after the fracturing flowback; pᵢ represents an initial coalbed pressure, MPa; p represents an average coal reservoir pressure, MPa; Cₛ represents a dissolution factor of coalbed methane in water, MPa⁻¹; B_{w} represents a volume factor of water, m³/m³; and C_{w} represents an isothermal compressibility of water, MPa⁻¹.

7. The method according to claim 1, wherein the cumulative adsorbed gas production of the target coalbed methane reservoir is calculated by a formula below:
$${G}_{p a} = V {ρ}_{c} \frac{{V}_{L} ( {p}_{i} + {p}_{c} )}{{p}_{L} + {p}_{i} + {p}_{c}} - V {ρ}_{c} \frac{{V}_{L} ( p + {p}_{c} )}{{p}_{L} + p + {p}_{c}}$$
wherein Gₚₐ represents a cumulative desorption quantity of adsorbed gas in the target coalbed methane reservoir into free pores, namely the cumulative adsorbed gas production, 10⁶ m³; ρ_{c} represents a coal rock density, t/m³; V_{L} represents Langmuir volume, m³/t; p_{L} represents Langmuir pressure, MPa; p_{c} represents a capillary pressure between target coalbed micropores and mesopores, MPa; pᵢ represents an initial coalbed pressure, MPa; and p represents an average coal reservoir pressure, MPa.

8. The method according to claim 1, wherein the daily free gas production of the target coalbed methane reservoir is calculated by a formula below:
$${q}_{pf , j} = \{ \begin{matrix}{10}^{6} \frac{{G}_{pf , j} - {G}_{pf , j - 1}}{1} , & {G}_{pf , j} > {G}_{pf , j - 1} \\ 0 , & {G}_{pf , j} \leq {G}_{pf , j - 1}\end{matrix}$$
wherein q_{pf,j} represents the daily free gas production of the target coalbed methane reservoir on a current day (day j), m³/d; G_{pf,j} represents the cumulative free gas production of the target coalbed methane reservoir on the current day (day j), 10⁶ m³; and G_{pf,j-1} represents the cumulative free gas production of the target coalbed methane reservoir on a previous day (day j-1), 10⁶ m³.

9. The method according to claim 1, wherein the daily adsorbed gas production is calculated by a formula below:
$${q}_{pa , j} = \{ \begin{matrix}{10}^{6} \frac{{G}_{p a , j} - {G}_{p a , j - 1}}{1} , & {G}_{p f , j} > {G}_{p f , j - 1} \\ {10}^{6} \frac{{G}_{p a , j} - {G}_{p a , j - 1} + {G}_{p f , j} - {G}_{p f , j - 1}}{1} , & {G}_{p f , j} \leq {G}_{p f , j - 1}\end{matrix}$$
wherein q_{pa,j} represents the daily adsorbed gas production of the target coalbed methane reservoir on a current day (day j), m³/d; G_{pa,j} represents the cumulative adsorbed gas production of the target coalbed methane reservoir on the current day (day j), 10⁶ m³; and G_{pa,j-1} represents the cumulative adsorbed gas production of the target coalbed methane reservoir on a previous day (day j-1), 10⁶ m³; and
the daily dissolved gas production is calculated by a formula below:
$${q}_{p s , j} = 1 {0}^{6} \frac{{G}_{p s , j} - {G}_{p s , j - 1}}{1}$$
wherein q_{ps,j} represents the daily dissolved gas production of the target coalbed methane reservoir on the current day (day j), m³/d; G_{ps,j} represents the cumulative dissolved gas production of the target coalbed methane reservoir on the current day (day j), 10⁶ m³; and G_{ps,j-1} represents the cumulative dissolved gas production of the target coalbed methane reservoir on the previous day (day j-1), 10⁶ m³.

10. A device for evaluating production proportions of different gases in a deep coalbed methane reservoir, comprising:
a memory configured to store instructions; and
a processor configured to invoke the instructions from the memory and implement the method for evaluating production proportions of different gases in a deep coalbed methane reservoir according to claim 1 when executing the instructions.

11. The device according to claim 10, wherein the step of calculating, after fracturing flowback and before production, a coalbed porosity and a coalbed water saturation at the beginning of production of the target coalbed methane reservoir after the fracturing flowback based on the physical property parameters of the target coalbed methane reservoir comprises:
obtaining a total fracturing fluid quantity during a fracturing process of the target coalbed methane reservoir and a cumulative flowback quantity during a flowback process;
calculating a net fracturing injection quantity based on the total fracturing fluid quantity and the cumulative flowback quantity;
calculating the coalbed porosity at the beginning of production of the target coalbed methane reservoir after the fracturing flowback based on a coalbed controlled volume, the net fracturing injection quantity, an initial porosity of the target coalbed methane reservoir, and a volume factor of water; and
calculating the coalbed water saturation based on the coalbed controlled volume, the initial porosity of the target coalbed methane reservoir, an initial water saturation, the net fracturing injection quantity, the volume factor of water, and the coalbed porosity at the beginning of production of the target coalbed methane reservoir after the fracturing flowback.

12. The device according to claim 10, wherein the step of calculating an average coalbed pressure value of the target coalbed methane reservoir each day based on the original adsorbed gas reserve, the original free gas reserve, and the original dissolved gas reserve of the target coalbed methane reservoir, a cumulative gas production, a cumulative water production, and a cumulative water influx each day, the coalbed porosity and the coalbed water saturation at the beginning of production of the target coalbed methane reservoir after the fracturing flowback, and the physical property parameters comprises:
establishing a material balance equation for adsorbed gas in adsorption pores according to the material balance principle for adsorbed gas desorption;
establishing a material balance equation for free gas and dissolved gas in free pores according to the material balance principle for the free gas and the dissolved gas;
establishing a material balance equation for the target coalbed methane reservoir according to the material balance equation for the adsorbed gas and the material balance equation for the free gas and the dissolved gas; and
substituting the original adsorbed gas reserve, the original free gas reserve, and the original dissolved gas reserve of the target coalbed methane reservoir, the cumulative gas production, the cumulative water production, the cumulative water influx, the coalbed porosity and the coalbed water saturation at the beginning of production of the target coalbed methane reservoir after the fracturing flowback, and the physical property parameters into the material balance equation for the target coalbed methane reservoir, and calculating the average coalbed pressure value by one of a Newton iteration method, a secant method, and a univariate cubic function calculation method.

13. The device according to claim 12, wherein the calculating the average coalbed pressure value by a univariate cubic function calculation method comprises:
establishing a univariate cubic function model according to the material balance equation for the target coalbed methane reservoir;
calculating a quadratic factor, a linear factor, and a constant term of the univariate cubic function model based on the original adsorbed gas reserve, the original free gas reserve, and the original dissolved gas reserve of the target coalbed methane reservoir, the cumulative gas production, the cumulative water production, and the cumulative water influx each day, the coalbed porosity and the coalbed water saturation at the beginning of production of the target coalbed methane reservoir after the fracturing flowback, and the physical property parameters; and
calculating the average coalbed pressure value according to the quadratic factor, the linear factor, and the constant term of the univariate cubic function model.

14. The device according to claim 10, wherein the cumulative free gas production of the target coalbed methane reservoir is calculated by a formula below:
$${G}_{p f} = {G}_{fi} - \frac{V {ϕ}_{i p} {Z}_{s c} {T}_{s c} p}{{p}_{s c} T Z} [ 1 - {S}_{w i p} - ( {C}_{p} + {S}_{w i p} {C}_{w} ) ( {p}_{i} - p ) - \frac{{W}_{e} - {W}_{p} {B}_{w}}{V {ϕ}_{i p}} + {C}_{a} ( \frac{{p}_{i} + {p}_{c}}{{p}_{L} + {p}_{i} + {p}_{c}} - \frac{p + {p}_{c}}{{p}_{L} + p + {p}_{c}} ) ]$$
wherein G_{pf} represents the cumulative free gas production of the target coalbed methane reservoir each day, 10⁶ m³; G_{fi} represents an original free gas reserve controlled by the target coalbed methane reservoir; Wₚ represents a cumulative water production of the target coalbed methane reservoir, 10⁶ m³; We represents a cumulative water influx during a production process, 10⁶ m³; φᵢₚ represents the coalbed porosity at the beginning of production of the target coalbed methane reservoir after the fracturing flowback; V represents a coalbed controlled volume, 10⁶ m³; S_{wip} represents the coalbed water saturation at the beginning of production of the target coalbed methane reservoir after the fracturing flowback; p_{sc} represents a pressure under standard conditions, MPa, with a value of 0.101325; T_{sc} represents a temperature under standard conditions, K, with a value of 293.15; Z_{sc} represents a natural gas deviation factor under standard conditions, dimensionless, with a value of 1; T represents a target coalbed temperature, K; Zᵢ represents a gas deviation factor under an initial coalbed pressure, dimensionless; p_{L} represents Langmuir pressure, MPa; p_{c} represents a capillary pressure between coalbed micropores and mesopores, MPa; pᵢ represents the initial coalbed pressure, MPa; p represents an average coal reservoir pressure, MPa; Z represents a gas deviation factor under an average coalbed pressure, dimensionless; Cₚ represents a pore volume compressibility, MPa⁻¹; C_{w} represents an isothermal compressibility of water, MPa⁻¹; Cₛ represents a dissolution factor of coalbed methane in water, MPa⁻¹; B_{w} represents a volume factor of water, m³/m³; and Ca represents a coal matrix shrinkage factor, dimensionless.

15. The device according to claim 10, wherein the cumulative dissolved gas production of the target coalbed methane reservoir is calculated by a formula below:
$${G}_{p s} = {G}_{s i} - V {ϕ}_{i p} {C}_{s} p [ {S}_{w i p} + {S}_{w i p} {C}_{w} ( {p}_{i} - p ) + \frac{{W}_{e} - {W}_{p} {B}_{w}}{V {ϕ}_{i p}} ]$$
wherein Gₚₛ represents the cumulative dissolved gas production of the target coalbed methane reservoir each day, 10⁶ m³; Gₛᵢ represents an original dissolved gas reserve controlled by the target coalbed methane reservoir; Wₚ represents a cumulative water production of the target coalbed methane reservoir, 10⁶ m³; We represents a cumulative water influx during a production process, 10⁶ m³; Pip represents the coalbed porosity at the beginning of production of the target coalbed methane reservoir after the fracturing flowback; V represents a coalbed controlled volume, 10⁶ m³; S_{wip} represents the coalbed water saturation at the beginning of production of the target coalbed methane reservoir after the fracturing flowback; pᵢ represents an initial coalbed pressure, MPa; p represents an average coal reservoir pressure, MPa; Cₛ represents a dissolution factor of coalbed methane in water, MPa⁻¹; B_{w} represents a volume factor of water, m³/m³; and C_{w} represents an isothermal compressibility of water, MPa⁻¹.

16. The device according to claim 10, wherein the cumulative adsorbed gas production of the target coalbed methane reservoir is calculated by a formula below:
$${G}_{p a} = V {ρ}_{c} \frac{{V}_{L} ( {p}_{i} + {p}_{c} )}{{p}_{L} + {p}_{i} + {p}_{c}} - V {ρ}_{c} \frac{{V}_{L} ( p + {p}_{c} )}{{p}_{L} + p + {p}_{c}}$$
wherein Gₚₐ represents a cumulative desorption quantity of adsorbed gas in the target coalbed methane reservoir into free pores, namely the cumulative adsorbed gas production, 10⁶ m³; ρ_{c} represents a coal rock density, t/m³; V_{L} represents Langmuir volume, m³/t; p_{L} represents Langmuir pressure, MPa; p_{c} represents a capillary pressure between target coalbed micropores and mesopores, MPa; pᵢ represents an initial coalbed pressure, MPa; and p represents an average coal reservoir pressure, MPa.

17. The device according to claim 10, wherein the daily free gas production of the target coalbed methane reservoir is calculated by a formula below:
$${q}_{pf , j} = \{ \begin{matrix}{10}^{6} \frac{{G}_{pf , j} - {G}_{pf , j - 1}}{1} , & {G}_{pf , j} > {G}_{pf , j - 1} \\ 0 , & {G}_{pf , j} \leq {G}_{pf , j - 1}\end{matrix}$$
wherein q_{pf,j} represents the daily free gas production of the target coalbed methane reservoir on a current day (day j), m³/d; G_{pf,j} represents the cumulative free gas production of the target coalbed methane reservoir on the current day (day j), 10⁶ m³; and G_{pf,j-1} represents the cumulative free gas production of the target coalbed methane reservoir on a previous day (day j-1), 10⁶ m³.

18. The device according to claim 10, wherein the daily adsorbed gas production is calculated by a formula below:
$${q}_{pa , j} = \{ \begin{matrix}{10}^{6} \frac{{G}_{p a , j} - {G}_{p a , j - 1}}{1} , & {G}_{pf , j} > {G}_{pf , j - 1} \\ {10}^{6} \frac{{G}_{p a , j} - {G}_{p a , j - 1} + {G}_{pf , j} - {G}_{pf , j - 1}}{1} , & {G}_{pf , j} \leq {G}_{pf , j - 1}\end{matrix}$$
wherein q_{pa,j} represents the daily adsorbed gas production of the target coalbed methane reservoir on a current day (day j), m³/d; G_{pa,j} represents the cumulative adsorbed gas production of the target coalbed methane reservoir on the current day (day j), 10⁶ m³; and G_{pa,j-1} represents the cumulative adsorbed gas production of the target coalbed methane reservoir on a previous day (day j-1), 10⁶ m³; and
the daily dissolved gas production is calculated by a formula below:
$${q}_{p s , j} = 1 {0}^{6} \frac{{G}_{p s , j} - {G}_{p s , j - 1}}{1}$$
wherein q_{ps,j} represents the daily dissolved gas production of the target coalbed methane reservoir on the current day (day j), m³/d; G_{ps,j} represents the cumulative dissolved gas production of the target coalbed methane reservoir on the current day (day j), 10⁶ m³; and G_{ps,j-1} represents the cumulative dissolved gas production of the target coalbed methane reservoir on the previous day (day j-1), 10⁶ m³.
